(19) United States
(12) Patent Application Publication
(10) Pub. No.: US 2008/0188542 A1
(43) Pub. Date: Aug. 7, 2008

(54) NOVEL INDOLE POLYMORPHS

(75) Inventors: Abdolsamad TADAYON, Kirkland (CA); Hsueh-Ling Wu, Edison, NJ (US); Mannching Sherry Ku, Thiells, NY (US); David Zenan Li, Princeton, NJ (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/026,235

(22) Filed: Feb. 5, 2008

Related U.S. Application Data
(60) Provisional application No. 60/899,473, filed on Feb. 5, 2007.

(51) Int. Cl. A61K 31/404; C07D 209/12; A61P 9/00; A61P 25/28

(52) U.S. Cl. 514/419; 548/493

(57) ABSTRACT

Novel polymorphs, pharmaceutical compositions containing novel polymorphs, methods of using novel polymorphs and methods of preparing novel polymorphs of [1-(4-tert-Butylbenzyl)-5-(3-methylphenyl)-1H-indol-3-yl](oxo)acetic acid are described herein.

### CROSS-REFERENCE TO RELATED APPLICATION

This application claims the benefit of U.S. Provisional Patent Application No. 60/899,473, filed Feb. 5, 2007, which is incorporated herein by reference in its entirety.

### FIELD OF THE INVENTION

This invention relates to novel indole polymorphs, pharmaceutical compositions containing novel indole polymorphs, and methods of using the novel indole polymorphs of this invention. In particular, the novel indole polymorphs of this invention are inhibitors of plasminogen activator inhibitor-1 (PAI-1) and are useful for the treatment of a wide variety of conditions including deep vein thrombosis, coronary heart disease, pulmonary fibrosis, cognition impairment, senility and Alzheimer's disease. BACKGROUND OF INVENTION

Plasminogen activator inhibitor-1 (PAI-1) is a major regulatory component of the plasminogen-plasmin system. PAI-1 is the principal physiologic inhibitor of both tissue type plasminogen activator (tPA) and urokinase type plasminogen activator (uPA). Elevated plasma levels of PAI-1 have been associated with thrombotic events as indicated by animal experiments (Krishnamurti, Blood, 69, 798 (1987); Reilly, Arteriosclerosis and Thrombosis, 11, 1276 (1991); Carmeliet, Journal of Clinical Investigations, 92, 2756 (1993)) and clinical studies (Rocha, Fibrinolysis, 8, 294, 1994; Aznar, Haemostasis 24, 243 (1994)). Antibody neutralization of PAI-1 activity resulted in promotion of endogenous thrombolysis and reperfusion (Biemond, Circulation, 91, 1175 (1995); Levi, Circulation 85, 305, (1992)). Elevated levels of PAI-1 have also been implicated in diseases of women such as polycystic ovary syndrome (Nordt, Journal of Clinical Endocrinology and Metabolism, 85, 4, 1563 (2000)) and bone loss induced by estrogen deficiency (Daci, Journal of Bone and Mineral Research, 15, 8, 1510 (2000)). Accordingly, agents that inhibit PAI-1 would be of utility in treating conditions originating from fibrinolytic disorder such as deep vein thrombosis, coronary heart disease, pulmonary fibrosis, polycystic ovary syndrome, etc.

PAI-1 inhibitors, by virtue of their ability to lead to the activation of plasmin, are predicted to reduce the levels of both soluble and aggregated forms of Aβ40/42 peptide by enhanced proteolytic clearance. Since Aβ40/42 comprise amyloid plaques associated with Alzheimer's disease, use of the novel polymorph of this invention is a promising candidate treatment for the prevention/treatment of Alzheimer's disease.

The compound [1-(4-tert-Butylbenzyl)-5-(3-methylphenyl)1H-indol-3-yl](oxo)acetic acid (I) is a PAI-1 inhibitor. The preparation and certain PAI-1 inhibition data of [1-(4-tert-Butylbenzyl)-5-(3-methylphenyl)-H-indol-3-yl](oxo)acetic acid have been described in U.S. Pat. No. 7,074,817 and the structure is shown below as formula 1.

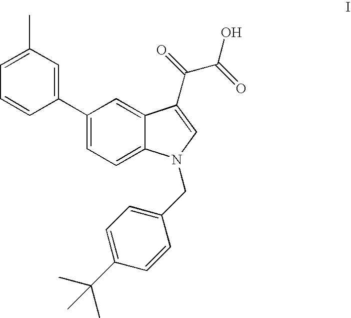

Compounds that exist as solids have the potential to exist in a crystalline state characterized by a repeating structural unit known as a lattice. In some cases, a given crystalline compound may be capable of forming more than one crystalline state, meaning that the same compound's solid state structure may differ between batches of prepared compound or even within a single batch of that compound. Solid compounds with no regular crystal lattice structure are commonly referred to as being “amorphous.” While some compounds have the capacity to exist in one or more crystalline states, others are amorphous only. Since the crystalline packing forces are part of the thermodynamic properties of the solid molecular substance, those packing forces can greatly affect physical parameters of the substance. For example, in the area of pharmaceuticals, it is well known that the particular crystalline form in which a drug substance is found can affect its solubility, stability, ease of formulation, processability, in vivo pharmacology, etc. In deciding which of the forms is preferable for a particular application, one generally studies each of the forms for its various attributes in view of numerous developmental considerations. In this regard, it is possible that one form may be preferred for a given use while another is preferred for a different use. Likewise, it is possible that each different form has advantages and disadvantages within a single use and the form chosen for development will represent the sum of all consideration. The ability to fully optimize a drug's properties, such as stability, ease of formulation, pharmacokinetics, etc, is highly sought after. As a result, there is an ongoing need for the discovery of new, crystalline forms of existing drug molecules. New polymorphs of [1-(4-tert-Butylbenzyl)-5-(3-methylphenyl)1H-indol-3-yl](oxo)acetic acid form are described herein.

### SUMMARY OF THE INVENTION

The present invention provides a polymorph A, B or C, or a combination thereof, of [1-(4-tert-Butylbenzyl)-5-(3-methylphenyl)1H-indol-3-yl](oxo)acetic acid characterized according to powder X-Ray diffraction data and DSC data as provided herein.

The present invention further provides compositions containing polymorph A, B or C, or a combination thereof of [1-(4-tert-Butylbenzyl)-5-(3-methylphenyl)1H-indol-3-yl](oxo)acetic acid.

The present invention further provides a method of preparing a polymorph A, B or C, or a combination thereof of [1-(4-tert-Butylbenzyl)-5-(3-methylphenyl)1H-indol-3-yl](oxo)acetic acid.

The present invention further provides a method for treatment of thrombosis or fibrinolytic impairment in a mammal comprising the administration of a composition comprising polymorph A, B or C, or a combination thereof of [1-(4-tert-Butylbenzyl)-5-(3-methylphenyl)1H-indol-3-yl](oxo)acetic acid to a mammal in need thereof.

In some embodiments, the thrombosis or fibrinolytic impairment is associated with formation of atherosclerotic plaques, venous or arterial thrombosis, myocardial ischemia, atrial fibrillation, deep vein thrombosis, coagulation syndromes, pulmonary fibrosis, cerebral thrombosis, thromboembolic complications of surgery or peripheral arterial occlusion.

The present invention further provides a method for treatment of myocardial ischemia in a mammal comprising the administration of a composition comprising polymorph A, B or C, or a combination thereof of [1-(4-tert-Butyl benzyl)-5-(3-methylphenyl)1H-indol-3-yl](oxo)acetic acid to a mammal in need thereof.

The present invention further provides a method for treatment of Alzheimer's disease in a mammal comprising the administration of a composition comprising polymorph A, B or C, or a combination thereof of [1-(4-tert-Butyl benzyl)-5-(3-methylphenyl)1H-indol-3-yl](oxo)acetic acid to a mammal in need thereof.

The present invention further provides a method of reducing amyloid beta levels in a mammal comprising the administration of a composition comprising a polymorph A, B or C, or a combination thereof of [1-(4-tert-Butylbenzyl)-5-(3-methylphenyl)-1H-indol-3-yl](oxo)acetic acid to a mammal in need thereof.

The present invention further provides a method of reducing amyloid beta levels in a mammal's brain comprising the administration of a composition comprising polymorph A, B or C, or a combination thereof of [1-(4-tert-Butylbenzyl)-5-(3-methylphenyl)-1H-indol-3-yl](oxo)acetic acid to a mammal in need thereof.

The present invention further provides a method of improving cognition in a mammal comprising the administration of a composition comprising polymorph A, B or C, or a combination thereof of [1-(4-tert-Butyl benzyl)-5-(3-methylphenyl)1H-indol-3-yl](oxo)acetic acid to a mammal in need thereof.

The present invention further provides a method of treating pre-senile or senile dementia in a mammal comprising the administration of composition comprising polymorph A, B or C, or a combination thereof of [1-(4-tert-Butyl benzyl)-5-(3-methylphenyl)1H-indol-3-yl](oxo)acetic acid to a mammal in need thereof.

The present invention further provides a method of treating amyotrophic lateral sclerosis in a mammal comprising the administration of a composition comprising polymorph A, B or C, or a combination thereof of [1-(4-tert-Butylbenzyl)-5-(3-methylphenyl)1H-indol-3-yl](oxo)acetic acid to a mammal in need thereof.

The present invention further provides for the use of a composition comprising polymorph A, B or C, or a combination thereof of [1-(4-tert-Butylbenzyl)-5-(3-methylphenyl)1H-indol-3-yl](oxo)acetic acid in the manufacture of a medicament useful for the treatment of Alzheimer's disease in a mammal.

The present invention further provides for the use of a composition comprising polymorph A, B or C of [1-(4-tert-Butylbenzyl)-5-(3-methylphenyl)1H-indol-3-yl](oxo)acetic acid in the manufacture of a medicament useful for the treatment of Alzheimer's disease in a mammal.

### BRIEF DESCRIPTION FOR THE DRAWINGS

FIG. 1 Depicts a powder X-ray diffraction pattern of [1-(4-tert-Butylbenzyl)-5-(3-methylphenyl)1H-indol-3-yl](oxo)acetic acid polymorph A, where the diffraction angle (2q) scan ranges from 5 to 30°.

FIG. 2 Depicts a differential scanning calorimetry (DSC) trace of [1-(4-tert-Butylbenzyl)-5-(3-methylphenyl)-1H-indol-3-yl](oxo)acetic acid polymorph A, using a scan range 37 up to 200° C., scan rate 10° C./min.

FIG. 3 Depicts a FT-Raman spectrum of polymorph A.

FIG. 4 Depicts a FT-IR spectrum of polymorph A.

FIG. 5 Depicts a solubility rate curve of polymorph A.

FIG. 6 Depicts a powder X-ray diffraction pattern of [1-(4-tert-Butylbenzyl)-5-(3-methylphenyl)1H-indol-3-yl](oxo)acetic acid polymorph B, where the diffraction angle (2 q) scan range 3-40°.

FIG. 7 Depicts a differential scanning calorimetry (DSC) trace of [1-(4-tert-Butylbenzyl)-5-(3-methylphenyl)1H-indol-3-yl](oxo)acetic acid polymorph B, using a scan range 25 up to 350° C., scan rate 10° C./min.

FIG. 8 Depicts a powder X-ray diffraction pattern of [1-(4-tert-Butylbenzyl)-5-(3-methylphenyl)1H-indol-3-yl](oxo)acetic acid polymorph C, where the diffraction angle (2 q) scan ranges from 5 to 30°.

FIG. 9 Depicts a differential scanning calorimetry (DSC) trace of [1-(4-tert-Butylbenzyl)-5-(3-methylphenyl)1H-indol-3-yl](oxo)acetic acid polymorph C for comparison, using a scan range 37 up to 200° C., scan rate 10° C./min.

### DETAILED DESCRIPTION OF THE INVENTION

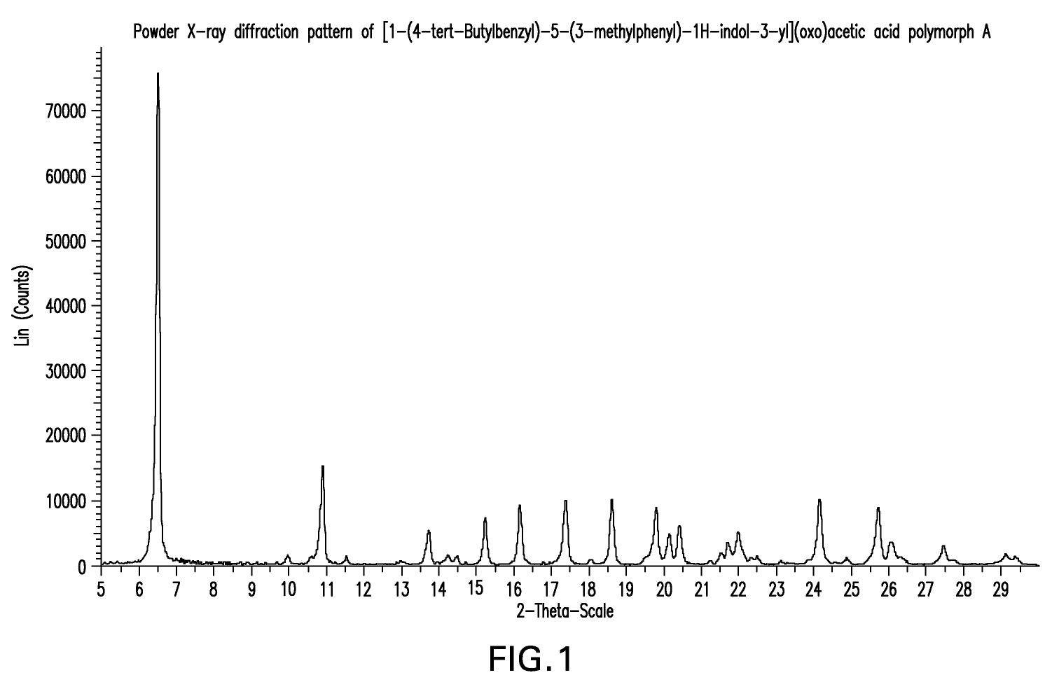

The present invention provides, inter alia, a polymorph of 1-(4-tert-Butylbenzyl)-5-(3-methylphenyl)1H-indol-3-yl](oxo)acetic acid, hereinafter referred to as “polymorph A,” which can be identified by one or more methods of solid state analytical chemistry. For example, polymorph A can be identified by the X-Ray powder diffraction which is provided in FIG. 1. Powder X-Ray diffraction data consistent with polymorph A is provided in Table 1 below.

TABLE 1
Powder diffraction data for polymorph A

| | Intensity, |
|---|---|
| Degree (2θ) | (% of the largest peak size) |
| 6.5 | 100.0 |
| 10.9 | 19.9 |
| 18.6 | 13.6 |
| 24.2 | 13.6 |
| 17.4 | 13.0 |
| 16.2 | 12.2 |
| 25.8 | 10.8 |
| 15.2 | 9.5 |
| 19.8 | 9.4 |
| 20.4 | 8.9 |
| 22.0 | 7.3 |
| 20.1 | 6.6 |
| 13.7 | 6.4 |
| 21.7 | 4.7 |
| 26.1 | 4.2 |
| 27.5 | 3.2 |
| 14.5 | 2.1 |
| 9.9 | 1.7 |
| 11.5 | 1.5 |
| 24.9 | 1.2 |
| 14.2 | 1.2 |

The present invention provides a method of treating a mammal, preferably a human, for a fibrinolytic impairment. In some embodiments, the fibrinolytic impairment is associated with the formation of atherosclerotic plaques, venous or arterial thrombosis, myocardial ischemia, atrial fibrillation, deep vein thrombosis, coagulation syndromes, pulmonary fibrosis, cerebral thrombosis, thromboembolic complications of surgery or peripheral arterial occlusion, comprising the administration of a therapeutically effective amount of a composition comprising a [1-(4-tert-Butylbenzyl)-5-(3-methylphenyl)1H-indol-3-yl](oxo)acetic acid polymorph A to a mammal in need thereof.

The present invention provides a method of treating a mammal, preferably a human, suffering from Alzheimer's disease, comprising the administration of a therapeutically effective amount of a composition comprising a [1-(4-tert-Butylbenzyl)-5-(3-methylphenyl)1H-indol-3-yl](oxo)acetic acid polymorph A to a mammal in need thereof.

The present invention provides a method of reducing amyloid beta levels in a mammal, preferably a human, suffering from Alzheimer's disease, comprising the administration of a therapeutically effective amount of a composition comprising polymorph A of [1-(4-tert-Butylbenzyl)-5-(3-methylphenyl)1H-indol-3-yl](oxo)acetic acid to a mammal in need thereof. In some embodiments, the methods of this invention reduce amyloid beta levels in the brain.

The present invention provides a method of improving cognition in a mammal, preferably a human, comprising the administration of a therapeutically effective amount of a composition comprising polymorph A of [1-(4-tert-Butylbenzyl)-5-(3-methylphenyl)-1H-indol-3-yl](oxo)acetic acid to a mammal in need thereof.

The present invention provides a method of treating pre-senile or senile dementia in a mammal, preferably a human, comprising the administration of therapeutically effective amount of a composition comprising polymorph A of [1-(4-tert-Butylbenzyl)-5-(3-methylphenyl)1H-indol-3-yl](oxo)acetic acid to a mammal in need thereof.

The present invention provides a method of treating amyotrophic lateral sclerosis in a mammal, preferably a human, comprising the administration of therapeutically effective amount of a composition comprising polymorph A of [1-(4-tert-Butylbenzyl)-5-(3-methylphenyl)1H-indol-3-yl](oxo)acetic acid to a mammal in need thereof.

The present invention provides a composition comprising polymorph A of [1-(4-tert-Butylbenzyl)-5-(3-methylphenyl)1H-indol-3-yl](oxo)acetic acid for the manufacture of a medicament useful for the treatment of Alzheimer's disease in a mammal, preferably a human.

The present invention provides a composition comprising polymorph A of [1-(4-tert-Butylbenzyl)-5-(3-methylphenyl)1H-indol-3-yl](oxo)acetic acid for the manufacture of a medicament useful for enhancing cognition in a mammal, preferably a human.

In some embodiments, the polymorph A of [1-(4-tert-Butylbenzyl)-5-(3-methylphenyl)1H-indol-3-yl](oxo)acetic acid has a powder X-ray diffraction pattern comprising characteristic peaks, in terms of 2θ, at about 6.5° and 10.9°. In further embodiments, the powder X-ray diffraction pattern further comprises characteristic peaks, in terms of 2θ, at about 18.6° and 24.2°. In yet further embodiments, the powder X-ray diffraction pattern further comprises characteristic peaks, in terms of 2θ, at about 17.4° and 16.2°. In still yet further embodiments, the powder X-ray diffraction pattern further comprises characteristic peaks, in terms of 2θ, at about 25.8° and 15.2°. In yet other embodiments, the powder X-ray diffraction pattern comprises characteristic peaks, in terms of 2θ, at about 6.5° and 10.9° and at least 5 additional characteristic peaks selected from 13.7°, 15.2°, 16.2°, 17.4°, 18.6°, 19.8°, 20.1°, 20.4°, 22.0°, 24.2°, and 25.8°. In yet other embodiments, the powder X-ray diffraction comprises characteristic peaks at about 6.5° and 10.9° and further comprises characteristic peaks, in terms of 2θ, at about 9.9°, 11.5°, 13.7°, 14.2°, 14.5°, 15.2°, 16.2°, 17.4°, 18.6°, 19.8°, 20.1°, 20.4°, 21.7°, 22.0°, 24.2°, 24.9°, 25.8°, 26.1°, and 27.5°. In some embodiments, polymorph A is characterized by an X-ray powder diffraction pattern substantially as shown in FIG. 1. The relative intensities of the peaks can vary, for example, upon sample preparation technique, sample mounting procedure, and particular instrument employed. Instrument variation and other factors may also affect the 2θ values.

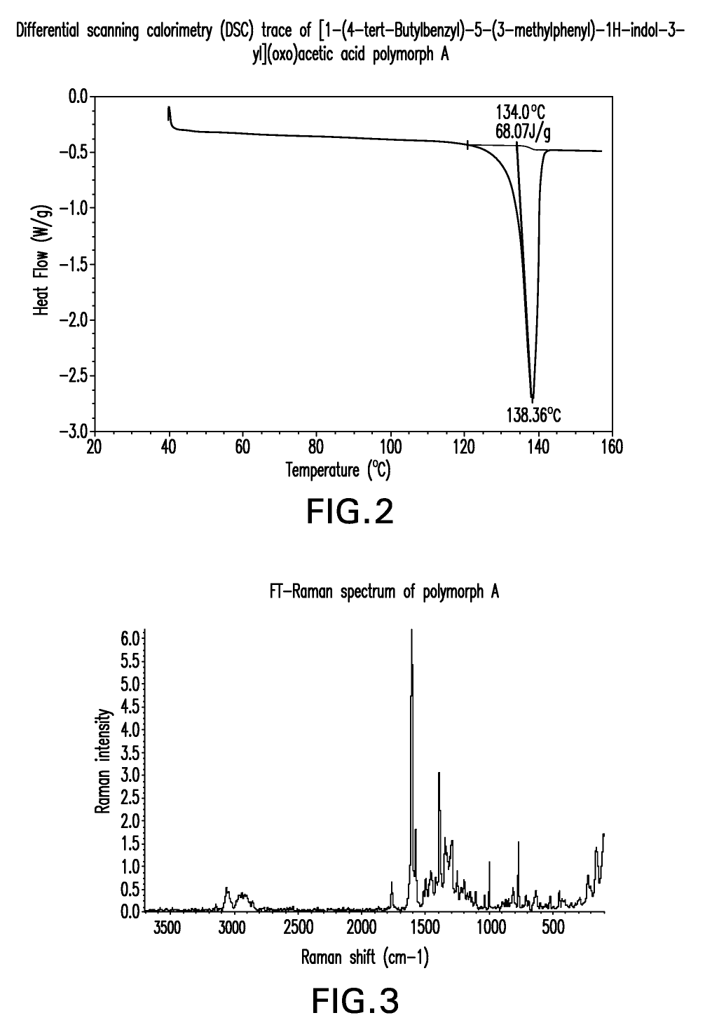

In certain embodiments, this invention describes polymorph A of [1-(4-tert-Butylbenzyl)-5-(3-methylphenyl)1H-indol-3-yl](oxo)acetic acid having a differential scanning calorimetry trace showing a melting peak greater than 134° C. In some embodiments, this invention describes polymorph A of [1-(4-tert-Butylbenzyl)-5-(3-methylphenyl)1H-indol-3-yl](oxo)acetic acid having a differential scanning calorimetry trace showing a melting peak greater than 136° C. In certain embodiments, this invention describes polymorph A of [1-(4-tert-Butylbenzyl)-5-(3-methylphenyl)-1H-indol-3-yl](oxo)acetic acid having a differential scanning calorimetry trace showing a melting peak greater than 138 C In certain embodiments, this invention describes polymorph A of [1-(4-tert-Butylbenzyl)-5-(3-methylphenyl)1H-indol-3-yl](oxo)acetic acid having a differential scanning calorimetry trace showing a melting peak at about 138° C. Polymorph A may be identified by its characteristic differential scanning calorimeter (DSC) trace such as that shown in FIG. 2. One of skill in the art appreciates that some variance in the melting peak can be expected with, for example, different rates of temperature during the scan, sample preparation and particular instrument employed. As a result, the values reported herein can vary by up to 4° C. in either direction.

In some embodiments, this invention describes a pharmaceutical composition comprising polymorph A of [1-(4-tert-Butylbenzyl)-5-(3-methylphenyl)1H-indol-3-yl](oxo)acetic acid. For purposes of understanding this invention, a pharmaceutical composition refers to the particular polymorph being referred to together with at least one pharmaceutically acceptable excipient. In certain embodiments, at least 3% by weight of total [1-(4-tert-Butylbenzyl)-5-(3-methylphenyl)1H-indol-3-yl](oxo)acetic acid in said pharmaceutical composition is polymorph A. In other embodiments, there is at least about 20% by weight of total [1-(4-tert-Butylbenzyl)-5-(3-methylphenyl)-1H-indol-3-yl](oxo)acetic acid polymorph A, or at least about 50%, or at least about 70%, or at least about 80%, or at least about 90%, or at least about 95%, or at least about 97%, or at least about 98%, or at least about 98.5%, or at least about 99%, or at least about 99.5%.

In some instances, the pharmaceutical compositions of this invention might be advantageously referred to by the amount of a particular polymorph in a composition relative to the weight of the composition itself. In certain embodiments, at least about 3% by weight of the pharmaceutical composition is polymorph A. In other embodiments, there is at least about 20% by weight of the pharmaceutical composition is polymorph A, or at least about 50%, or at least about 70%, or at least about 80%, or at least about 90%, or at least about 95%, or at least about 97%, or at least about 98%, or at least about 98.5%, or at least about 99%, or at least about 99.5%.

In some embodiments, this invention describes a mixture containing one or more polymorphs of the invention. Such mixtures will comprise one or more polymorphs of the invention, together with additional substances, such as chemical impurities, additives, polymorphs other than the one specified, other active chemical moieties, etc. A mixture does not refer to the reference polymorph and a pharmaceutically acceptable excipient since, for purposes of this invention, that type of combination is referred to as a pharmaceutical composition.

In some embodiments, this invention describes a mixture comprising the polymorph A, wherein said polymorph A comprises at least about 3% by weight of total [1-(4-tert-Butylbenzyl)-5-(3-methylphenyl)1H-indol-3-yl](oxo)acetic acid in said mixture. In some embodiments, polymorph A comprises at least about 10% by weight of total [1-(4-tert-Butylbenzyl)-5-(3-methylphenyl)-1H-indol-3-yl](oxo)acetic acid in said mixture; or about 50%; or about 90%; or about 95%; or about 99%.

In some embodiments, this invention describes a mixture comprising the polymorph A, wherein said polymorph A comprises at least about 3% by weight of total weight of said mixture. In some embodiments, polymorph A comprises at least about 10% by weight of total weight in said mixture; or about 50%; or about 90%; or about 95%; or about 99%.

Polymorph A of [1-(4-tert-Butylbenzyl)-5-(3-methylphenyl)-1H-indol-3-yl](oxo)acetic acid can be distinguished from other polymorphic forms (for example, B and C) of [1-(4-tert-Butylbenzyl)-5-(3-methylphenyl)1H-indol-3-yl](oxo)acetic acid, by for example, a comparison of melting points and X-Ray Powder diffraction (see Table 2).

TABLE 2
Some comparative data between three polymorphs (A, B and C) of
[1-(4-tert-Butylbenzyl)-5-(3-methylphenyl)-1H-indol-3-yl](oxo)acetic acid

| Measurement | Polymorph A | Polymorph B | Polymorph C |
|---|---|---|---|
| Melting Point | 138° C. | 131° C. | 82° C. |
| Melting onset | 134° C. | 128° C. | 74° C. |
| DSC | Single Melting | Single Melting | Broad Single |
| | Endotherm | Endotherm | Melting |
| | 138.36° C. | 131.28° C. | Endotherm |
| | | | 82.24° C. |
| X-Ray Powder | 6.5°, 10.9°, | 5.2°, 10.3°, | 5.4°, 6.8°, |
| (2θ) | 18.6° | 10.8° | 13.5° |

Polymorph A may also be characterized by FT-Raman as shown in FIG. 3. Selected peaks from the FT-Raman spectrum for polymorph A are listed below in Table 3. In certain embodiments of this invention, the polymorph A of [1-(4-tert-Butylbenzyl)-5-(3-methylphenyl)-1H-indol-3-yl](oxo)acetic acid of this invention comprises characteristic peaks at approximately 1610 cm⁻¹ and 1393 cm⁻¹ of the FT-Raman. In some embodiments of this invention, polymorph A of [1-(4-tert-Butylbenzyl)-5-(3-methylphenyl)-1H-indol-3-yl](oxo)acetic acid further comprises characteristic peaks at approximately 1576 cm ⁻¹ and 1347 cm⁻of the FT-Raman. In yet other embodiments of this invention, the polymorph A of [1-(4-tert-Butylbenzyl)-5-(3-methylphenyl)-1H-indol-3-yl](oxo)acetic acid further comprises characteristic peaks at approximately 1292 cm⁻¹ and 773 cm⁻¹ of the FT-Raman. In still yet other embodiments of this invention, the polymorph A of [1-(4-tert-Butylbenzyl)-5-(3-methylphenyl)-1H-indol-3-yl](oxo)acetic acid further comprises characteristic peaks at approximately 1000 cm⁻¹ and 160 cm⁻¹ of the FT-Raman. In another embodiment, the polymorph A of [1-(4-tert-Butylbenzyl)-5-(3-methylphenyl)1H-indol-3-yl](oxo)acetic acid of this invention comprises peaks at 1610 cm⁻¹, 1393 cm⁻¹, and three additional peaks selected from 1576 cm⁻¹, 1347 cm⁻¹, 1292 cm⁻¹, 1000 cm⁻¹, 773 cm⁻¹ and 160 cm⁻¹ of the FT-Raman. In some aspects, the polymorph A of [1-(4-tert-Butylbenzyl)-5-(3-methylphenyl)-1H-indol-3-yl](oxo)acetic acid of this invention has a FT-Raman substantially as shown in FIG. 3.

TABLE 3
FT-Raman spectrum of polymorph A

| Wavenumbers (cm⁻¹) | Raman intensity |
|---|---|
| 1609.65 | 6.211 |
| 1392.69 | 3.103 |
| 1576.04 | 1.817 |
| 1347.29 | 1.629 |
| 1291.95 | 1.595 |
| 772.84 | 1.548 |
| 160.44 | 1.393 |
| 999.56 | 1.142 |
| 1249.92 | 0.931 |
| 1458.52 | 0.894 |
| 224.97 | 0.811 |
| 1496.16 | 0.760 |
| 1196.13 | 0.700 |
| 1766.22 | 0.670 |
| 3063.99 | 0.536 |
| 815.11 | 0.535 |
| 450.44 | 0.478 |
| 635.09 | 0.459 |
| 1108.81 | 0.438 |
| 2943.20 | 0.416 |
| 1037.78 | 0.387 |
| 712.78 | 0.362 |
| 524.37 | 0.359 |

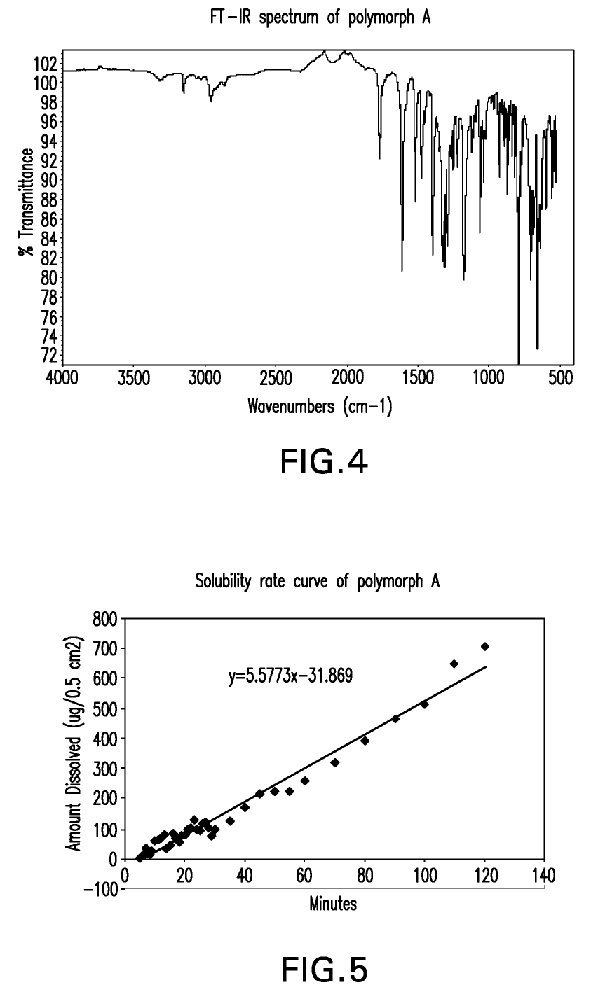

Polymorph A may also be characterized by FT-IR as reproduced in FIG. 4. Selected peaks from the FT-IR spectrum for polymorph A are listed below in Table 4. In certain embodiments of this invention, the polymorph A of [1-(4-tert-Butylbenzyl)-5-(3-methylphenyl)-1H-indol-3-yl](oxo)acetic acid of this invention comprises characteristic peaks at approximately 788 cm⁻¹ and 655 cm⁻¹ of the FT-IR. In some embodiments of this invention, the polymorph A of [1-(4-tert-Butylbenzyl)-5-(3-methylphenyl)1H-indol-3-yl](oxo)acetic acid further comprises characteristic peaks at approximately 1173 cm ⁻¹ and 703 cm⁻¹ of the FT-IR. In yet other embodiments of this invention, the polymorph A of [1-(4-tert-Butylbenzyl)-5-(3-methylphenyl)-1H-indol-3-yl](oxo)acetic acid further comprises characteristic peaks at approximately 1610 cm⁻¹ and 1311 cm⁻¹ of the FT-IR. In still yet other embodiments of this invention, the polymorph A of [1-(4-tert-Butylbenzyl)-5-(3-methylphenyl)1H-indol-3-yl](oxo)acetic acid further comprises characteristic peaks at approximately 1394 cm⁻¹ and 1323 cm⁻of the FT-IR. In still yet another embodiment of this invention, polymorph A of [1-(4-tert-Butylbenzyl)-5-(3-methylphenyl)-1H-indol-3-yl](oxo)acetic acid further comprises characteristic peaks at approximately 1289 cm⁻¹ and 636 cm⁻¹ of the FT-IR. In another embodiment, the polymorph A of [1-(4-tert-Butylbenzyl)-5-(3-methylphenyl)1H-indol-3-yl](oxo)acetic acid of this invention comprises peaks at 788 cm⁻¹ and 655 cm⁻¹, and three additional peaks selected from 1610 cm⁻¹, 1394 cm⁻¹, 1323 cm⁻¹, 1311 cm⁻¹, 1289 cm⁻¹, 1173 cm⁻, 703 cm⁻¹ and 636 cm⁻¹ of the FT-IR. In some aspects, the polymorph A of [1-(4-tert-Butylbenzyl)-5-(3-methylphenyl)-1H-indol-3-yl](oxo)acetic acid of this invention has an FT-IR substantially as shown in FIG. 4.

TABLE 4
FT-IR Spectrum of Crystalline polymorph A

| Wavenumbers (cm⁻¹) | % Transmittance |
|---|---|
| 788.31 | 70.750 |
| 655.36 | 72.346 |
| 1173.40 | 79.558 |
| 703.15 | 79.411 |
| 1609.75 | 80.397 |
| 1310.80 | 80.893 |
| 1323.18 | 81.552 |
| 1394.14 | 82.177 |
| 636.44 | 82.822 |
| 1289.36 | 82.936 |
| 645.45 | 83.522 |
| 715.06 | 84.260 |
| 685.63 | 83.985 |
| 1059.65 | 84.115 |
| 678.28 | 84.942 |
| 796.17 | 86.616 |
| 599.05 | 86.656 |
| 1517.14 | 87.621 |
| 559.24 | 87.968 |
| 772.18 | 87.948 |
| 868.32 | 88.232 |
| 546.00 | 89.143 |
| 527.59 | 89.049 |
| 1037.53 | 89.737 |
| 1472.52 | 89.776 |
| 814.30 | 90.094 |
| 927.86 | 90.183 |
| 1249.01 | 90.834 |
| 1220.94 | 91.237 |
| 1267.27 | 92.076 |
| 1766.07 | 92.094 |
| 835.07 | 92.365 |
| 1120.53 | 92.732 |
| 758.22 | 93.290 |
| 884.58 | 93.302 |
| 850.24 | 93.960 |
| 899.54 | 93.752 |
| 535.64 | 94.277 |
| 1449.54 | 95.737 |
| 911.58 | 96.774 |
| 964.17 | 97.166 |
| 2956.99 | 97.919 |
| 3149.11 | 98.812 |

The polymorph A of [1-(4-tert-Butylbenzyl)-5-(3-methylphenyl)-1H-indol-3-yl](oxo)acetic acid of this invention can also be characterized by its solubility rate which is shown in FIG. 5. The solubility rate intrinsic dissolution was conducted in pH 7.5 phosphate buffer medium using USP apparatus 2 (paddle) at 50 rpm. The dissolution rate may be influenced by disk rotation speed and other conditions. The solubility of polymorph A of [1-(4-tert-Butylbenzyl)-5-(3- methylphenyl)-1H-indol-3-yl](oxo)acetic acid has been determined in a number of solvents and the results are listed in Table 5. The solubilities listed in Table 5 are based on measurements taken at equilibrium.

TABLE 5
Solubility of polymorph A at room temperature

| Solvent | Solubility of Polymorph A | |
|---|---|---|
| Deionized Water | 0.024 | mg/mL |
| 2% Polysorbate80/0.5% Methylcellulose | 1.09 | mg/mL |
| Polysorbate 80 | >200 | mg/g |
| PEG 200 | >100 | mg/g |
| PEG 400 | >100 | mg/g |
| Propylene Glycol | 7.1 | mg/mL |
| Propylene Carbonate | 103.2 | mg/mL |
| Capryol 90 | 73.8 | mg/g |
| Cremophore EL | 290 | mg/g |
| Labrasol | >160 | mg/g |
| Phosal 53 MCT | 311 | mg/g |
| Ethanol | 12.24 | mg/mL |
| Acetone | 277 | mg/mL |
| DMSO | >500 | mg/mL |

The compound of this invention containing [1-(4-tert-Butylbenzyl)-5-(3-methylphenyl)1H-indol-3-yl](oxo)acetic acid, may be prepared via methods known to one of ordinary skill in the art. For example, the preparation of [1-(4-tert-Butylbenzyl)-5-(3-methylphenyl)-1H-indol-3-yl](oxo)acetic acid was disclosed in U.S. Pat. No. 7,074,817 as example 34.

The preparation of polymorph A of [1-(4-tert-Butylbenzyl)-5-(3-methylphenyl)1H-indol-3-yl](oxo)acetic acid is described herein as follows. In certain embodiments, this invention describes a method of preparing polymorph A comprising slurrying or crystallization of [1-(4-tert-Butylbenzyl)-5-(3-methylphenyl)1H-indol-3-yl](oxo)acetic acid in one or more aromatic hydrocarbons, one or more polar non-protic solvents, or one or more dialkylethers, or combinations thereof. In some embodiments, the aromatic hydrocarbon is selected from benzene, toluene, ethylbenzene, ortho-xylene, meta-xylene or para-xylene or combinations thereof. In some embodiments, said aromatic hydrocarbon comprises toluene. In certain embodiments, said dialkyl ether solvent is selected from propylethyl ether, isopropyl-ethyl ether, t-butyl methyl ether, and sec-butyl methyl ether. In some embodiments, said dialkylether is t- butyl methyl ether. In certain embodiments, the polar non-protic solvent is acetonitrile.

In some embodiments, this invention describes a method of preparing polymorph A comprising dissolution of [1-(4-tert-Butylbenzyl)-5-(3-methylphenyl)1H-indol-3-yl](oxo)acetic acid in acetonitrile at a temperature sufficient for dissolution, cooling to from about 15° C. to about 30° C., treating with water and collecting polymorph A.

In some embodiments, this invention describes a method of preparing polymorph A comprising dissolution of [1-(4-tert-Butylbenzyl)-5-(3-methylphenyl)1H-indol-3-yl](oxo)acetic acid in toluene at a temperature sufficient for dissolution, addition of a non-aromatic hydrocarbon and collecting polymorph A. In certain embodiments, the solution is cooled prior to collecting polymorph A.

In certain embodiments, this invention describes a method of preparing polymorph A comprising dissolution of [1-(4-tert-Butylbenzyl)-5-(3-methylphenyl)1H-indol-3-yl](oxo)acetic acid in t-butyl methyl ether at a temperature sufficient for dissolution, addition of a non-aromatic hydrocarbon, seeding with polymorph A, and collecting the polymorph A.

In some embodiments, additional non-aromatic hydrocarbon was added after seeding followed by cooling of the solution to a temperature between about 15° C. to about 30° C.

### EXAMPLES

Presented below are some representative examples of the invention but the present invention should not be construed as being limited to the examples presented.

### Preparation of [1 -(4-tert-Butylbenzyl)-5-(3-methylphenyl)1H-indol-3-yl](oxo)acetic acid as Crystalline Polymorph A

### Example 1

200 mg of [1-(4-tert-Butylbenzyl)-5-(3-methylphenyl)-1H-indol-3-yl](oxo)acetic acid was dissolved in 0.5 ml acetonitrile at 55° C. in a 5 ml vial fixed with a magnetic stirrer. The solution was cooled to room temperature in 40 min. 1 ml water was added to the solution. The suspension was stirred for 1 hr. The suspension was filtered and dried in an oven at 50° C. under vacuum overnight. The analysis showed that the solids are polymorph A (melting point=134° C.).

### Example 2

161 mg of [1-(4-tert-Butylbenzyl)-5-(3-methylphenyl)-1H-indol-3-yl](oxo)acetic acid was added to 0.5 ml toluene and dissolved at 55° C. in a 5 ml vial fixed with a magnetic stirrer and 2 ml heptane was added; the solution crystallized and the suspension stirred for 30 min. The temperature was decreased to room temperature and the suspension was stirred for 4 hrs. The suspension was filtered, and dried in an oven at 60° C. under vacuum over night. The analysis showed that the solids are polymorph A (XRD, melting point=137° C.).

### Example 3

2.5 gr of [1-(4-tert-Butylbenzyl)-5-(3-methylphenyl)1H-indol-3-yl](oxo)acetic acid was added to 10 ml t-butyl methyl ether, the temperature kept at 53° C. until all solids dissolved. 10 ml heptane was added; the solution was seeded with crystals of polymorph A and stirred for 2 hrs. An additional 10 ml heptane was added in 1 hr, stirred for 1 hr, cooled to room temperature in 2 hrs, and stirred overnight (The analysis showed that the solids was polymorph A; melting point of about 132° C.).

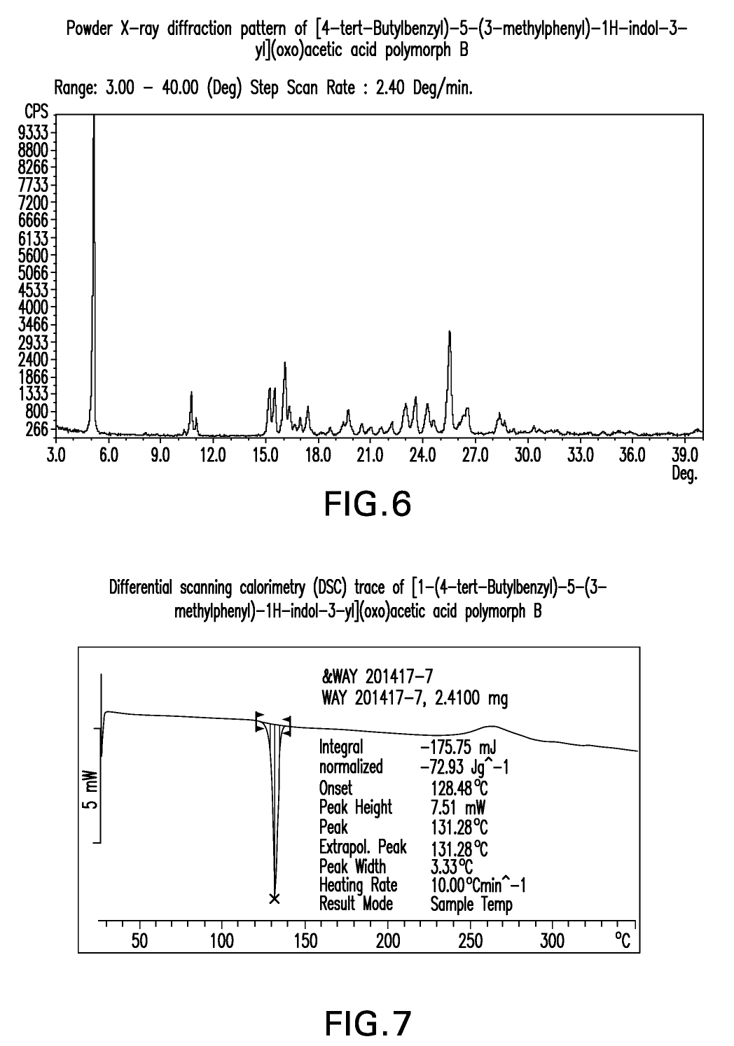

The present invention also provides, inter alia, a polymorph B of 1-(4-tert-Butylbenzyl)-5-(3-methylphenyl)1H-indol-3-yl](oxo)acetic acid, herein referred to as polymorph B, which can be identified by one or more methods of solid state analytical chemistry. For example, polymorph B can be identified by the X-Ray powder diffraction which is provided in FIG. 6. Powder X-Ray diffraction data consistent with polymorph B is provided in Table 6 below.

TABLE 6
Powder diffraction data for polymorph B

| | Intensity, |
|---|---|
| Degree (2θ) | (% of the largest peak size) |
| 5.2 | 100 |
| 25.6 | 32.5 |
| 16.1 | 21.0 |
| 15.5 | 13.6 |
| 15.2 | 13.5 |
| 10.8 | 13.4 |
| 23.6 | 10.1 |
| 24.3 | 8.6 |
| 23.0 | 8.2 |
| 26.6 | 8.2 |
| 17.4 | 7.9 |
| 16.4 | 7.7 |
| 19.7 | 7.4 |
| 28.4 | 5.6 |
| 11.1 | 5.0 |
| 17.0 | 4.6 |
| 22.3 | 3.5 |
| 24.6 | 3.4 |
| 28.7 | 3.4 |
| 19.5 | 3.1 |
| 29.5 | 2.9 |
| 16.6 | 1.9 |
| 21.0 | 1.8 |
| 18.7 | 1.7 |
| 30.4 | 1.7 |
| 21.6 | 1.5 |
| 10.3 | 1.5 |
| 30.7 | 1.0 |
| 31.4 | 1.0 |
| 31.7 | 1.0 |
| 35.9 | 0.9 |

The present invention provides a method of treating a mammal, preferably a human, for a fibrinolytic impairment. In some embodiments, the fibrinolytic impairment is associated with the formation of atherosclerotic plaques, venous or arterial thrombosis, myocardial ischemia, atrial fibrillation, deep vein thrombosis, coagulation syndromes, pulmonary fibrosis, cerebral thrombosis, thromboembolic complications of surgery or peripheral arterial occlusion, comprising the administration of a therapeutically effective amount of a composition comprising a [1-(4-tert-Butylbenzyl)-5-(3-methylphenyl)1H-indol-3-yl](oxo)acetic acid polymorph B to a mammal in need thereof.

The present invention provides a method of treating a mammal, preferably a human, suffering from Alzheimer's disease, comprising the administration of a therapeutically effective amount of a composition comprising a [1-(4-tert-Butylbenzyl)-5-(3-methylphenyl)-1H-indol-3-yl](oxo)acetic acid polymorph B to a mammal in need thereof.

The present invention provides a method of reducing amyloid beta levels in a mammal, preferably a human, suffering from Alzheimer's disease, comprising the administration of a therapeutically effective amount of a composition comprising polymorph B of [1-(4-tert-Butylbenzyl)-5-(3-methylphenyl)1H-indol-3-yl](oxo)acetic acid to a mammal in need thereof. In some embodiments, the methods of this invention reduce amyloid beta levels in the brain.

The present invention provides a method of improving cognition in a mammal, preferably a human, comprising the administration of a therapeutically effective amount of a composition comprising a polymorph B of [1-(4-tert-Butylbenzyl)-5-(3-methylphenyl)1H-indol-3-yl](oxo)acetic acid to a mammal in need thereof.

The present invention provides a method of treating pre-senile or senile dementia in a mammal, preferably a human, comprising the administration of a therapeutically effective amount of a composition comprising polymorph B of [1-(4-tert-Butylbenzyl)-5-(3-methylphenyl)-1H-indol-3-yl](oxo)acetic acid to a mammal in need thereof.

The present invention provides a method of treating amyotrophic lateral sclerosis in a mammal, preferably a human, comprising the administration of therapeutically effective amount of a composition comprising polymorph B of [1-(4-tert-Butylbenzyl)-5-(3-methylphenyl)1H-indol-3-yl](oxo)acetic acid to a mammal in need thereof.

The present invention provides a composition comprising polymorph B of [1-(4-tert-Butylbenzyl)-5-(3-methylphenyl)1H-indol-3-yl](oxo)acetic acid for the manufacture of a medicament useful for the treatment of Alzheimer's disease in a mammal, preferably a human.

The present invention provides a composition comprising polymorph B of [1-(4-tert-Butylbenzyl)-5-(3-methylphenyl)1H-indol-3-yl](oxo)acetic acid for the manufacture of a medicament useful for enhancing cognition in a mammal, preferably a human.

In some embodiments, the polymorph B of [1-(4-tert-Butylbenzyl)-5-(3-methylphenyl)1H-indol-3-yl](oxo)acetic acid has a powder X-ray diffraction pattern comprising characteristic peaks, in terms of 2θ, at about 5.2° and 25.6°. In further embodiments, the powder X-ray diffraction pattern further comprises characteristic peaks, in terms of 2θ, at about 15.5° and 16.1°. In yet further embodiments, the powder X-ray diffraction pattern further comprises characteristic peaks, in terms of 2θ, at about 10.8° and 15.2°. In still yet further embodiments, the powder X-ray diffraction pattern further comprises characteristic peaks, in terms of 2θ, at about 23.0°, 23.6°, 24.3° and 26.6°. In yet other embodiments, the powder X-ray diffraction pattern comprises characteristic peaks, in terms of 2θ, at about 5.2° and 25.6° and at least 5 additional characteristic peaks selected from 16.1°, 15.5°, 15.2°, 10.8°, 23.0°, 23.6°, 24.3° and 26.6°. In yet other embodiments, the powder X-ray diffraction comprises characteristic peaks at about 5.2° and 25.6° and further comprises characteristic peaks, in terms of 2θ, at about 10.3°, 10.8°, 11.1°, 15.2°, 15.5°, 16.1°, 16.4°, 16.6°, 17.0°, 17.4°, 18.7°, 19.5°, 19.7°, 21.0°, 21.6°, 22.3°, 23.0°, 23.6°, 24.3°, 24.6°, 26.6°, 28.4°, 28.7°, 29.5°, 30.4°, 30.7°, 31.4°, 31.7°, and 35.9°. In some embodiments, the polymorph B is characterized by an X-ray powder diffraction pattern substantially as shown in FIG. 6. The relative intensities of the peaks can vary, for example, upon sample preparation technique, sample mounting procedure, and particular instrument employed. Instrument variation and other factors may also affect the 20 values.

In certain embodiments, this invention describes polymorph B of [1-(4-tert-Butylbenzyl)-5-(3-methylphenyl)1H-indol-3-yl](oxo)acetic acid having a differential scanning calorimetry trace showing a melting peak at about 131° C. Polymorph B may thus be identified by its characteristic differential scanning calorimeter (DSC) trace such as that shown in FIG. 7. One of skill in the art appreciates that some variance in the melting peak can be expected with, for example, different rates of temperature during the scan, sample preparation and particular instrument employed. As a result, the values reported herein can vary by up to 4° C. in either direction.

In some embodiments, this invention describes a pharmaceutical composition comprising polymorph B of [1-(4-tert-Butylbenzyl)-5-(3-methylphenyl)1H-indol-3-yl](oxo)acetic acid. For purposes of understanding this invention, a pharmaceutical composition refers to the particular polymorph being referred to together with at least one pharmaceutically acceptable excipient. In certain embodiments, at least about 3% by weight of total [1-(4-tert-Butylbenzyl)-5-(3-methylphenyl)-1H-indol-3-yl](oxo)acetic acid in said pharmaceutical composition is Form B. In other embodiments, there is at least about 20% by weight of total [1-(4-tert-Butylbenzyl)-5-(3-methylphenyl)-1H-indol-3-yl](oxo)acetic acid is polymorph B, or at least about 50%, or at least about 70%, or at least about 80%, or at least about 90%, or at least about 95%, or at least about 97%, or at least about 98%, or at least about 98.5%, or at least about 99%, or at least about 99.5%.

In certain embodiments, at least about 3% by weight of the pharmaceutical composition is polymorph B. In other embodiments, there is at least about 20% by weight of the pharmaceutical composition is polymorph B, or at least about 50%, or at least about 70%, or at least about 80%, or at least about 90%, or at least about 95%, or at least about 97%, or at least about 98%, or at least about 98.5%, or at least about 99%, or at least about 99.5%.

In some embodiments, this invention describes a mixture comprising the polymorph B, wherein said polymorph B comprises at least about 3% by weight of total [1-(4-tert-Butylbenzyl)-5-(3-methylphenyl)1H-indol-3-yl](oxo)acetic acid in said mixture. In some embodiments, polymorph B comprises at least about 10% by weight of total [1-(4-tert-Butylbenzyl)-5-(3-methylphenyl)-1H-indol-3-yl](oxo)acetic acid in said mixture; or about 50%; or about 90%; or about 95%; or about 99%.

In some embodiments, this invention describes a mixture comprising the polymorph B, wherein said polymorph B comprises at least about 3% by weight of total weight of said mixture. In some embodiments, polymorph B comprises at least about 10% by weight of total weight in said mixture; or about 50%; or about 90%; or about 95%; or about 99%.

Polymorph B of [1-(4-tert-Butylbenzyl)-5-(3-methylphenyl)-1H-indol-3-yl](oxo)acetic acid can be distinguished from other polymorphic forms (for example, A and C) of [1-(4-tert-Butylbenzyl)-5-(3-methylphenyl)1H-indol-3-yl](oxo)acetic acid, by for example, a comparison of melting points and X-Ray Powder diffraction (see Table 2, supra).

The preparation of polymorph B of [1-(4-tert-Butylbenzyl)-5-(3-methylphenyl)1H-indol-3-yl](oxo)acetic acid can be accomplished, for example, as described herein.

In some embodiments, polymorph B of [1-(4-tert-Butylbenzyl)-5-(3-methylphenyl)-1H-indol-3-yl](oxo)acetic acid is prepared by dissolution of [1-(4-tert-Butylbenzyl)-5-(3-methylphenyl)1H-indol-3-yl](oxo)acetic acid in one or more aromatic hydrocarbons followed by evaporation of solvent. In certain embodiments, the aromatic hydrocarbon comprises toluene. In some embodiments, the dissolution of [1-(4-tert-Butylbenzyl)-5-(3-methylphenyl)-1H-indol-3-yl](oxo)acetic acid in the aromatic hydrocarbon solvent is accomplished by heating the solvent at greater than about 50° C. In some embodiments, the dissolution of [1-(4-tert-Butylbenzyl)-5-(3-methylphenyl)1H-indol-3-yl](oxo)acetic acid in the aromatic hydrocarbon solvent is accomplished by heating the solvent at between about 60° C. and about 80° C.

Presented below are some representative examples of the invention but the present invention should not be construed as being limited to the examples presented.

### Preparation of [1-(4-tert-Butylbenzyl)-5-(3-methylphenyl)1H-indol-3-yl](oxo)acetic Acid as Polymorph B

### Example 4

78 g of 2-[1-[4-(tert-Butyl)benzyl]-5-(3-methylphenyl)-1H-indol-3-yl]-2-oxoacetic acid was dissolved in toluene (155 mL) by heating to about 70° C. in a 500 mL Erlenmeyer flask that was stirred with a magnetic stirrer. The stirring was ceased, and the flask was sealed with a septum stopper inserted with 2 needles (19 G). The mixture was allowed to stand at room temperature for 2.5 days to permit slow evaporation without seeding. After 2.5 days some crystals were observed and the septum stopper was removed. The mixture was allowed to stand for another 6 hours. The crystals formed gradually and were filtered. After drying in the open air overnight, the crystals were chopped up and dried in vacuo at 70° C. for 2 days to afford the title compound as a light yellow crystals (57.1 g), m.p. 133-133.5° C. DSC and XRD showed that crystals are of polymorph B.

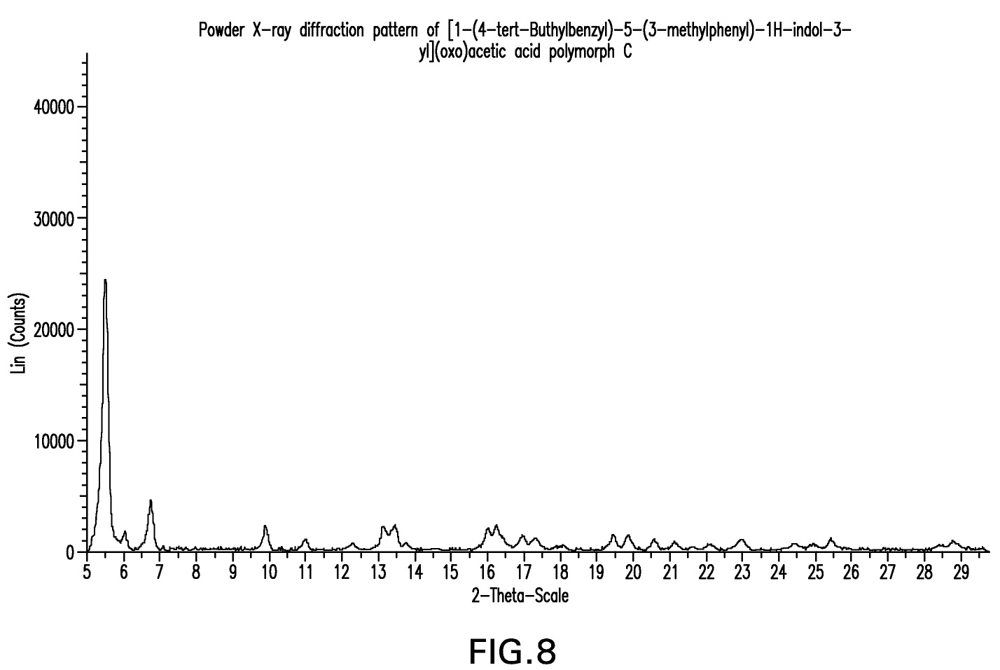

The present invention also provides, inter alia, a polymorph of 1-(4-tert-Butylbenzyl)-5-(3-methylphenyl)-1H-indol-3-yl](oxo)acetic acid, herein referred to as polymorph C, which can be identified by one or more methods of solid state analytical chemistry. For example, polymorph C can be identified by the X-Ray powder diffraction which is provided in FIG. 8. Powder X-Ray diffraction data consistent with polymorph C is provided in Table 7 below.

TABLE 7
Powder diffraction data for polymorph C

| | Intensity, |
|---|---|
| Degree (2θ) | (% of the largest peak size) |
| 5.4 | 100 |
| 6.8 | 16.7 |
| 13.5 | 9.0 |
| 9.9 | 8.5 |
| 6.0 | 8.3 |
| 16.2 | 6.9 |
| 13.2 | 5.6 |
| 17.3 | 5.6 |
| 19.9 | 4.9 |
| 20.6 | 4.9 |
| 17.0 | 4.2 |
| 19.5 | 4.2 |
| 23.0 | 3.8 |
| 10.9 | 3.4 |
| 21.2 | 2.6 |
| 24.5 | 2.0 |
| 22.1 | 1.9 |

The present invention provides a method of treating a mammal, preferably a human, for a fibrinolytic impairment. In some embodiments, the fibrinolytic impairment is associated with the formation of atherosclerotic plaques, venous or arterial thrombosis, myocardial ischemia, atrial fibrillation, deep vein thrombosis, coagulation syndromes, pulmonary fibrosis, cerebral thrombosis, thromboembolic complications of surgery or peripheral arterial occlusion, comprising the administration of a therapeutically effective amount of a composition comprising a [1-(4-tert-Butylbenzyl)-5-(3-methylphenyl)1H-indol-3-yl](oxo)acetic acid polymorph C to a mammal in need thereof.

The present invention provides a method of treating a mammal, preferably a human, suffering from Alzheimer's disease, comprising the administration of a therapeutically effective amount of a composition comprising a [1-(4-tert-Butylbenzyl)-5-(3-methylphenyl)1H-indol-3-yl](oxo)acetic acid polymorph C to a mammal in need thereof.

The present invention provides a method of reducing amyloid beta levels in a mammal, preferably a human, suffering from Alzheimer's disease, comprising the administration of a therapeutically effective amount of a composition comprising a polymorph C of [1-(4-tert-Butylbenzyl)-5-(3-methylphenyl)1H-indol-3-yl](oxo)acetic acid to a mammal in need thereof. In some embodiments, the methods of this invention reduce amyloid beta levels in the brain.

The present invention provides a method of improving cognition in a mammal, preferably a human, comprising the administration of a therapeutically effective amount of a composition comprising a polymorph C of [1-(4-tert-Butylbenzyl)-5-(3-methylphenyl)1H-indol-3-yl](oxo)acetic acid to a mammal in need thereof.

The present invention provides a method of treating pre-senile or senile dementia in a mammal, preferably a human, comprising the administration of a therapeutically effective amount of a composition comprising a polymorph C of [1-(4-tert-Butylbenzyl)-5-(3-methylphenyl)1H-indol-3-yl](oxo)acetic acid to a mammal in need thereof.

The present invention provides a method of treating amyotrophic lateral sclerosis in a mammal, preferably a human, comprising the administration of a therapeutically effective amount of a composition comprising a polymorph C of [1-(4-tert-Butylbenzyl)-5-(3-methylphenyl)1H-indol-3-yl](oxo)acetic acid to a mammal in need thereof.

The present invention provides a composition comprising a polymorph C of [1-(4-tert-Butylbenzyl)-5-(3-methylphenyl)1H-indol-3-yl](oxo)acetic acid for the manufacture of a medicament useful for the treatment of Alzheimer's disease in a mammal, preferably a human.

The present invention provides a composition comprising a polymorph C of [1-(4-tert-Butylbenzyl)-5-(3-methylphenyl)1H-indol-3-yl](oxo)acetic acid for the manufacture of a medicament useful for enhancing cognition in a mammal, preferably a human.

In some embodiments, polymorph C of [1-(4-tert-Butylbenzyl)-5-(3-methylphenyl)-1H-indol-3-yl](oxo)acetic acid has a powder X-ray diffraction pattern comprising characteristic peaks, in terms of 2θ, at about 5.4° and 6.8°. In further embodiments, the powder X-ray diffraction pattern further comprises characteristic peaks, in terms of 2θ, at about 13.5° and 9.9°. In yet further embodiments, the powder X-ray diffraction pattern further comprises characteristic peaks, in terms of 2θ, at about 16.2° and 6.0°. In still yet further embodiments, the powder X-ray diffraction pattern further comprises characteristic peaks, in terms of 2θ, at about 20.6°, 19.9°, 17.3° and 13.2°. In yet other embodiments, the powder X-ray diffraction pattern comprises characteristic peaks, in terms of 2θ, at about 5.4° and 6.8° and at least 5 additional characteristic peaks selected from 6.0°, 13.2°, 13.5°, 16.2°, 17.3°, 19.9° and 20.6°. In yet other embodiments, the powder X-ray diffraction comprises characteristic peaks at about 5.4° and 6.8° and further comprises characteristic peaks, in terms of 2θ, at about 6.0°, 9.9°, 10.9°, 13.2°, 13.5°, 16.2°, 17.0°, 17.3°, 19.5°, 19.9°, 20.6°, 21.2°, 22.1°, 23.0°, 24.5°.

In some embodiments, polymorph C is characterized by an X-ray powder diffraction pattern substantially as shown in FIG. 8. The relative intensities of the peaks can vary, for example, upon sample preparation technique, sample mounting procedure, and particular instrument employed. Instrument variation and other factors may also affect the 2θ values.

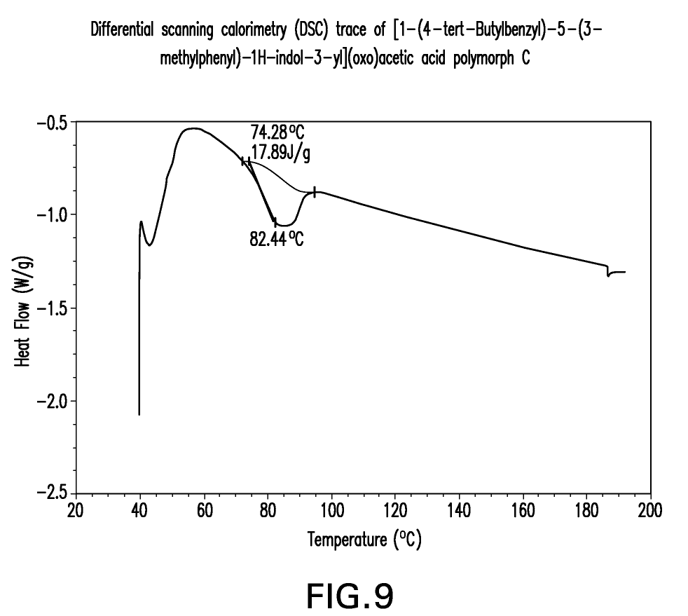

In certain embodiments, this invention describes a polymorph C of [1-(4-tert-Butylbenzyl)-5-(3-methylphenyl)1H-indol-3-yl](oxo)acetic acid having a differential scanning calorimetry trace showing a melting peak at about 82.24° C. Polymorph C may thus be identified by its characteristic differential scanning calorimeter (DSC) trace such as that shown in FIG. 9. One of skill in the art appreciates that some variance in the melting peak can be expected with, for example, different rates of temperature during the scan, sample preparation and particular instrument employed. As a result, the values reported herein can vary by up to 4° C. in either direction.

In some embodiments, this invention describes a pharmaceutical composition comprising polymorph C of [1-(4-tert-Butylbenzyl)-5-(3-methylphenyl)1H-indol-3-yl](oxo)acetic acid. In certain embodiments, at least about 3% by weight of total [1-(4-tert-Butylbenzyl)-5-(3-methylphenyl)-1H-indol-3-yl](oxo)acetic acid in said pharmaceutical composition is Form C. In other embodiments, there is at least about 20% by weight of total [1-(4-tert-Butylbenzyl)-5-(3-methylphenyl)1H-indol-3-yl](oxo)acetic acid is polymorph C, or at least about 50%, or at least about 70%, or at least about 80%, or at least about 90%, or at least about 95%, or at least about 97%, or at least about 98%, or at least about 98.5%, or at least about 99%, or at least about 99.5%.

In certain embodiments, at least about 3% by weight of the pharmaceutical composition is Form C. In other embodiments, there is at least about 20% by weight of the pharmaceutical composition is polymorph C, or at least about 50%, or at least about 70%, or at least about 80%, or at least about 90%, or at least about 95%, or at least about 97%, or at least about 98%, or at least about 98.5%, or at least about 99%, or at least about 99.5%.

In some embodiments, this invention describes a mixture comprising the polymorph C, wherein said polymorph C comprises at least about 3% by weight of total [1-(4-tert-Butylbenzyl)-5-(3-methylphenyl)1H-indol-3-yl](oxo)acetic acid in said mixture. In some embodiments, polymorph C comprises at least about 10% by weight of total [1-(4-tert-Butylbenzyl)-5-(3-methylphenyl)-1H-indol-3-yl](oxo)acetic acid in said mixture; or about 50%; or about 90%; or about 95%; or about 99%.

In some embodiments, this invention describes a mixture comprising the polymorph C, wherein said polymorph C comprises at least about 3% by weight of total weight of said mixture. In some embodiments, polymorph C comprises at least about 10% by weight of total weight in said mixture; or about 50%; or about 90%; or about 95%; or about 99%.

Polymorph C of [1-(4-tert-Butylbenzyl)-5-(3-methylphenyl)-1H-indol-3-yl](oxo)acetic acid can be distinguished from other polymorphic forms (for example, A and B) of [1-(4-tert-Butylbenzyl)-5-(3-methylphenyl)1H-indol-3-yl](oxo)acetic acid, by for example, a comparison of melting points and X-Ray Powder diffraction (see Table 2, supra).

The preparation of polymorph C of [1-(4-tert-Butylbenzyl)-5-(3-methylphenyl)1H-indol-3-yl](oxo)acetic acid can be accomplished, for example, as described herein.

In some embodiments, a polymorph C of [1-(4-tert-Butylbenzyl)-5-(3-methylphenyl)-1H-indol-3-yl](oxo)acetic acid is prepared by dissolution of [1-(4-tert-Butylbenzyl)-5-(3-methylphenyl)-1H-indol-3-yl](oxo)acetic acid in a solvent comprising an alcohol followed by crystallization and collection of polymorph C. In some embodiments, the solution comprising the alcohol is heated to effect dissolution and cooled to effect crystallization. In certain embodiments, the solution comprising alcohol is treated with a non-aromatic hydrocarbon counter solvent prior to collecting the desired polymorph. In some embodiments, the alcohol is ethanol. In certain embodiments, the non-aromatic hydrocarbon is heptane.

### Preparation of [1-(4-tert-Butylbenzyl)-5-(3-methylphenyl)1H-indol-3-yl](oxo)acetic Acid as Polymorph C

### Example 5

201 gr. of [1-(4-tert-Butylbenzyl)-5-(3-methylphenyl)1H-indol-3-yl](oxo)acetic acid was added to 4 ml of ethanol (containing 0.5% toluene) in a 5 ml vial with a magnetic stirrer. The solution temperature increased to 65° C. 0.5 ml of the solution was transferred to a 2 ml vial and the temperature was gradually cooled to room temperature. The solution crystallized; 0.5 ml heptanes was added and stirred overnight. The suspension was filtered and dried under vacuum at 50° C. DSC and XRD showed that crystals are of polymorph C.

In this disclosure, the term “polymorphs” is used to mean polymorph A, B or C, or a combination thereof, unless otherwise specified. Similarly, the term “polymorph compositions” refers to a composition containing polymorph A, B or C, or a combination thereof and at least one pharmaceutically acceptable excipient, unless otherwise specified.

The methods of this invention contemplate treatment as well as prevention of disease states where applicable. Accordingly, reference to treatment will normally mean treatment and/or prevention where appropriate to the context. Likewise, reference to treatment of a disease state refers to both treating the disease and/or arresting the progression of symptoms of the disease. Likewise, reference to treatment also includes prevention of symptoms of the disease state where appropriate to the context.

[1-(4-tert-Butylbenzyl)-5-(3-methylphenyl)1H-indol-3-yl](oxo)acetic acid, described herein as its polymorphs, and compositions comprising 1-(4-tert-Butylbenzyl)-5-(3-methylphenyl)-1H-indol-3-yl](oxo)acetic acid polymorphs, are inhibitors of the serine protease inhibitor PAI-1, and are therefore useful in the treatment, inhibition, prevention or prophylaxis in a mammal, preferably in a human, of those processes which involve the production and/or action of PAI-1 (a “PAI-1 related disorder”). Thus, the polymorph compositions of the invention are useful in the treatment or prevention of noninsulin dependent diabetes mellitus and cardiovascular, ocular or kidney disease caused by such condition, and prevention of thrombotic events associated with coronary artery and cerebrovascular disease. These polymorph compositions are also useful for inhibiting the disease process involving the thrombotic and prothrombotic states which include, but are not limited to, formation of atherosclerotic plaques, venous and arterial thrombosis, myocardial ischemia, atrial fibrillation, deep vein thrombosis, coagulation syndromes, pulmonary fibrosis, cerebral thrombosis, thromboembolic complications of surgery (such as joint replacement), and peripheral arterial occlusion. These polymorph compositions are also useful in treating ischemic events, such as stroke, associated with or resulting from atrial fibrillation.

The polymorph compositions of the invention may also be used in the treatment of diseases associated with extracellular matrix accumulation, including, but not limited to, renal fibrosis, chronic obstructive pulmonary disease, polycystic ovary syndrome, restenosis, renovascular disease and organ transplant rejection.

The polymorph compositions of the invention may also be used in the treatment of malignancies, and diseases associated with neoangiogenesis (such as diabetic retinopathy and age-related macular degeneration).

The polymorph compositions in the invention may also be used in conjunction with and following processes or procedures involving maintaining blood vessel patency, including vascular surgery, vascular graft and stent patency, organ, tissue and cell implantation and transplantation.

The polymorph compositions in the invention may also be useful in the treatment of inflammatory diseases, septic shock and the vascular damage associated with infections.

The polymorph compositions of the invention are useful for the treatment of blood and blood products used in dialysis, blood storage in the fluid phase, especially ex vivo platelet aggregation. The present compositions may also be added to human plasma during the analysis of blood chemistry in hospital settings to determine the fibrinolytic capacity thereof.

The polymorph compositions in the present invention may also be used in combination with prothrombolytic, fibrinolytic and anticoagulant agents.

The polymorph compositions of the present invention may also be used to treat cancer including, but not limited to, breast and ovarian cancer, and as imaging agents for the identification of metastatic cancers.

The polymorph compositions of the invention may also be used in the treatment of Alzheimer's disease. This method may also be characterized as the inhibition of plasminogen activator by PAI-1 in a mammal, particularly a human, experiencing or subject to Alzheimer's disease. This method may also be characterized as a method of increasing or normalizing levels of plasmin concentration in a mammal, particularly those experiencing or subject to Alzheimer's disease.

Not wishing to be bound by theory, the polymorph compositions of this invention provide a method of reducing beta-amyloid in a mammal, preferably a human. This reduction may be systemic or in some cases the reduction of beta amyloid may be primarily localized to the CNS or brain of the subject. Thus, the compositions of this invention are useful tools for the lowering of beta amyloid in a subject where such lowering in beta amyloid is determined to be a desired outcome. The compositions of this invention are useful in the treatment of amyotrophic lateral sclerosis (“ALS”).

The polymorph compositions of this invention are also useful for preserving or enhancing cognition in a mammal, preferably a human.

The polymorphs and compositions containing those polymorphs of this invention are useful in the manufacture of medicaments useful for the methods of this invention. For example, the polymorphs and compositions of this invention are useful in the manufacture of medicaments for the treatment of Alzheimer's disease. The polymorphs and compositions of this invention are useful in the manufacture of medicaments useful for enhancing cognition in a mammal.

The polymorph compositions of the invention may be used for the treatment of myelofibrosis with myeloid metaplasia by regulating stromal cell hyperplasia and increases in extracellular matrix proteins.

The polymorph compositions of the invention may also be used in conjunction with protease inhibitor—containing highly active antiretroviral therapy (HAART) for the treatment of diseases which originate from fibrinolytic impairment and hyper-coagulability of HIV-1 infected patients receiving such therapy.

The polymorph compositions of the invention may be used for the treatment of diabetic nephropathy and renal dialysis associated with nephropathy.

The polymorph compositions of the invention may be used to treat cancer, septicemia, obesity, insulin resistance, proliferative diseases such as psoriasis, improve coagulation homeostasis, treat cerebrovascular diseases, microvascular disease, hypertension, dementia, osteoporosis, arthritis, asthma, heart failure, arrhythmia, angina, as a hormone replacement agent, treate, prevente or reverse progression of atherosclerosis, Alzheimer's disease, osteoporosis, and osteopenia; reduce inflammatory markers, reduce C-reactive protein, prevent or treat low grade vascular inflammation, stroke, dementia, coronary heart disease, for primary and secondary prevention of myocardial infarction, stable and unstable angina, for primary prevention of coronary events, for secondary prevention of cardiovascular events, peripheral vascular disease, peripheral arterial disease, and acute vascular syndromes, reduce the risk of undergoing a myocardial revascularization procedure, treat microvascular diseases such as nephropathy, neuropathy, retinopathy and nephrotic syndrome, hypertension, Type 1 and 2 diabetes and related diseases, hyperglycemia, hyperinsulinemia, malignant lesions, premalignant lesions, gastrointestinal malignancies, liposarcomas and epithelial tumors, proliferative diseases such as psoriasis, improve coagulation homeostasis, and/or endothelial function, and treat all forms of cerebrovascular diseases.

The polymorph compositions of this invention comprise [1-(4-tert-Butylbenzyl)-5-(3-methylphenyl)-1H-indol-3-yl](oxo)acetic acid polymorphs together with a pharmaceutical excipient. If it is desired, the compositions of this invention may comprise [1-(4-tert-Butylbenzyl)-5-(3-methylphenyl)-1H-indol-3-yl](oxo)acetic acid polymorphs together with [1-(4-tert-Butylbenzyl)-5-(3-methylphenyl)1H-indol-3-yl](oxo)acetic acid that is amorphous.

For use in the methods of this invention, [1-(4-tert-Butylbenzyl)-5-(3-methylphenyl)-1H-indol-3-yl](oxo)acetic acid polymorphs can be administered as pharmaceutical compositions by any method known in the art for administering therapeutic drugs, including oral, buccal, topical, systemic (e.g., transdermal, intranasal, or by suppository), or parenteral (e.g., intramuscular, subcutaneous, intrathecal, intra-articular, peri-spinal or intravasuclar injection). Depending on the formulation used, it is possible that the [1-(4-tert-Butylbenzyl)-5-(3-methylphenyl)1H-indol-3-yl](oxo)acetic acid polymorphs used in the methods of this invention may not be present in that particular due to dissolution or interconversion in the administering vehicle, for example, where an IV formulation is contemplated for use, or where certain emulsions or syrups are contemplated.

Compositions can take the form of tablets, pills, capsules, semisolids, powders, sustained release formulations, solutions, suspensions, emulsions, syrups, elixirs, aerosols, or any other appropriate compositions; and comprise [1-(4-tert-Butylbenzyl)-5-(3-methylphenyl)-1H-indol-3-yl](oxo)acetic acid polymorphs in combination with at least one pharmaceutically acceptable excipient. Suitable excipients are well known to persons of ordinary skill in the art, and they, and the methods of formulating the compositions, can be found in such standard references as Alfonso AR: Remington's Pharmaceutical Sciences, 17th ed., Mack Publishing Company, Easton Pa., 1985, the disclosure of which is herein incorporated by reference.

In some embodiments of the present invention, the [1-(4-tert-Butylbenzyl)-5-(3-methylphenyl)1H-indol-3-yl](oxo)acetic acid polymorphs can be administered either singly or in combination with at least one other active moiety. For example, the [1-(4-tert-Butylbenzyl)-5-(3-methylphenyl)-1H-indol-3-yl](oxo)acetic acid polymorphs of the present invention can also be administered with at least one other conventional therapeutic agent for the disease being treated.

Aqueous suspensions of [1-(4-tert-Butylbenzyl)-5-(3-methylphenyl)1H-indol-3-yl](oxo)acetic acid polymorphs can be prepared and administered in admixture with excipients suitable for the manufacture of aqueous suspensions. Such excipients can include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents, and one or more sweetening agents, such as sucrose, aspartame or saccharin. Formulations can be adjusted for osmolarity.

Oil suspensions can be formulated by suspending [1-(4-tert-Butylbenzyl)-5-(3-methylphenyl)-1H-indol-3-yl](oxo)acetic acid polymorphs in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

In certain preferred modes, pharmaceutical compositions comprising [1-(4-tert-Butylbenzyl)-5-(3-methylphenyl)1H-indol-3-yl](oxo)acetic acid polymorphs are liquid or emulsified dosage formulations especially suitable to the dosing of mammals. In such a formulation, [1-(4-tert-Butyl benzyl)-5-(3-methylphenyl)1H-indol-3-yl](oxo)acetic acid polymorphs may be combined with one or more solubilizers/emulsifiers. Preferred compound concentration ranges in such a formulation can range from 0.01% to 10% w/w of the composition. Some useful solubilizers/emulsifiers are glycerol-polyethylene glycol esters of fatty acids. For example, a Cremophor® EL. If desired, a flavor masking agent may be used in the context of this invention or alternative, hydrogenated Cremophors such as Cremophor® RH40 may be used.

As mentioned before, the emulsion or liquefied form of the compositions of this invention may comprise more than one solubilizer/emulsifier. Thus, for example, the compositions useful for the methods of the invention may comprise ester of a hydroxylated fatty acid such as a 12- or 15-hydroxystearate, polyglycol mono- and di-esters of 12-hydroxystearic acid wherein said polyglycol mono- and di-esters of 12-hydroxystearic acid can further comprise from 20 to 40% or about 30% free polyethylene glycol. In some embodiments, the solubilizer/emulsifier is Solutol HS15 or macrogol 15 hydroxystearate. Other useful solubilizer/surfactants include propylene glycol mono-esters such as propylene glycol dioleate, 2-hydroxypropyl stearate, 2-hydroxypropyl laurate, propylene glycol monostearate, propylene glycol oleate, propylene glycol distearate, propylene glycol dicaprylate, propylene glycol monolaurate, propylene glycol dilaurate, polypropylene glycol (17) dioleate, propyleneglycol monolaurate, Propylene glycol monomyristate, dipropylene glycol dipelargonate, propylene glycol monocaprylate, polypropylene glycol monobutyl ether oleate, propylene glycol dipelargonate, propylene glycol didecanoate, dipropylene glycol dipelargonate, propylene glycol bis(9,10-epoxystearate), propylene glycol monoisostearate, propylene glycol diundecanoate and propylene glycol monocaprylate (Capryol® 90). Additional solubilizer/emulsifiers may be contemplated alone, or in combination such as, for example, non-ionic surfactants. Some preferred non-ionic surfactants include polysorbates. For example, polysorbate 80 is a useful surfactant for compositions of this invention. The solubilizer/emulsifiers may be further combined with additional excipients if so desired. In a preferred embodiment of this invention, [1-(4-tert-Butylbenzyl)-5-(3-methylphenyl)-1H-indol-3-yl](oxo)acetic acid polymorphs are present in from 0.01% to 10% w/w, a emulsifier/solubilizer (e.g. Cremophor®) is present in from 36% to 40% w/w, another solubilizer/emulsifier (e.g. Solutol®) is present in from 36% to 40% w/w, another solubilizer/emulsifier is present in from 18% to 20% and the remainder comprises additional excipients.

The compound of choice, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for parenteral administration, such as, for example, by intraarticular (in the joints), intravascular (e.g., intravenous and intra-arterial), intramuscular, intradermal, intraperitoneal, intrathecal, peri-spinal and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. Where the compounds are sufficiently soluble they can be dissolved directly in normal saline with or without the use of suitable organic solvents, such as propylene glycol or polyethylene glycol.

Dispersions of the finely divided compounds can be made-up in aqueous starch or sodium carboxymethyl cellulose solution, or in suitable oil, such as arachis oil. These formulations can be sterilized by conventional, well known sterilization techniques. The formulations can contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of [1-(4-tert-Butylbenzyl)-5-(3-methylphenyl)1H-indol-3-yl](oxo)acetic acid polymorphs thereof in these formulations can vary widely, and will be selected primarily based on fluid volumes, viscosities, body weight of the patient, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol. The formulations can be presented in unit-dose or multi-dose sealed containers, such as ampules and vials.

Injection solutions and suspensions can be prepared from sterile powders, granules, and tablets of the kind previously described.

[1-(4-tert-Butylbenzyl)-5-(3-methylphenyl)1H-indol-3-yl](oxo)acetic acid polymorphs suitable for use in the practice of this invention can be administered orally. The amount of a compound of the present invention in the composition can vary widely depending on the type of composition, size of a unit dosage, kind of excipients, and other factors well known to those of ordinary skill in the art. In general, the final composition can comprise, for example, from about 0.000001 percent by weight (% w) to about 50% w of [1-(4-tert-Butylbenzyl)-5-(3-methylphenyl)-1H-indol-3-yl](oxo)acetic acid polymorphs, preferably about 3.33% w to about 33.33% w, with the remainder being the excipient or excipients.

Pharmaceutical formulations for oral administration can be formulated using pharmaceutically acceptable carriers well known in the art in dosages suitable for oral administration. Such carriers enable the pharmaceutical formulations to be formulated in unit dosage forms as tablets, pills, powder, dragees, capsules, liquids, lozenges, gels, syrups, slurries, suspensions, etc., suitable for ingestion by the patient. Formulations suitable for oral administration can consist of: (a) liquid solutions, such as an effective amount of the packaged nucleic acid suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions.

Pharmaceutical preparations for oral use can be obtained, for example, through combination of the compounds of the present invention with a solid excipient, optionally grinding a resulting mixture, and processing the mixture of granules, after adding suitable additional compounds, if desired, to obtain tablets or dragee cores. Suitable solid excipients are carbohydrate or protein fillers and include, but are not limited to sugars, including lactose, sucrose, mannitol, or sorbitol; starch from corn, wheat, rice, potato, or other plants; cellulose such as methyl cellulose, hydroxymethyl cellulose, hydroxypropylmethyl-cellulose or sodium carboxymethylcellulose; and gums including arabic and tragacanth; as well as proteins such as gelatin and collagen. If desired, disintegrating or solubilizing agents can be added, such as the cross-linked polyvinyl pyrrolidone, agar, alginic acid, or a salt thereof, such as sodium alginate. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

In certain preferred embodiments, [1-(4-tert-Butylbenzyl)-5-(3-methylphenyl)-1H-indol-3-yl](oxo)acetic acid polymorphs suitable for solid dosage form for oral delivery is present in a concentration of from about 3.33% to about 33.33% w/w, together with additional excipients including one or more surfactants in a collective range of from about 1% to about 25% w/w, one or more binders in a collective range of from about 1% to about 35% w/w, one or more disintegrants in a collective range of from about 2% to about 8% w/w, one or more glidants in a collective range of from about 0.01% to about 5% w/w, one or more lubricants in a collective range of from about 0.01% to about 5% w/w with additional excipients making up the remainder. As an example of a preferred embodiment falling within the above listed component specification is a composition suitable for oral dosage comprising [1-(4-tert-Butylbenzyl)-5-(3-methylphenyl)-1H-indol-3-yl](oxo)acetic acid polymorphs in a range of from about 3.33% to about 33.33% w/w, about 5% w/w TPGS (d-alpha-tocopheryl polyethylene glycol 1000 succinate), about 5% sodium lauryl sulfate, about 15% microcrystalline cellulose, about 5% croscarmellose sodium, about 0.5% colloidal silicon dioxide, about 0.5% magnesium stearate and the remainder as filler. In one preferred embodiment, this composition is filled into a capsule.

The [1-(4-tert-Butyl benzyl)-5-(3-methylphenyl)1H-indol-3-yl](oxo)acetic acid polymorphs of the present invention can also be administered in the form of suppositories for intra-cavity (i.e., intravaginal or rectal) administration of the drug. These formulations can be prepared by mixing the drug with a suitable non-irritating excipient which is solid at ordinary temperatures but liquid at body temperatures and will therefore melt in the cavity to release the drug. Such materials include cocoa butter and polyethylene glycols.

The [1-(4-tert-Butylbenzyl)-5-(3-methylphenyl)1H-indol-3-yl](oxo)acetic acid polymorphs of the present invention can be administered by intranasal, intraocular, intravaginal, and intrarectal routes including suppositories, insufflation, powders and aerosol formulations (for examples of suitable inhalants, see Rohatagi, J. Clin. Pharmacol. 35:1187-1193, 1995; Tjwa, Ann. Allergy Asthma Immunol. 75:107-111, 1995, the disclosure of which is herein incorporated by reference).

The [1-(4-tert-Butyl benzyl)-5-(3-methylphenyl)1H-indol-3-yl](oxo)acetic acid polymorphs of the present invention can be delivered transdermally, by a topical route, formulated as applicator sticks, solutions, suspensions, emulsions, gels, creams, ointments, pastes, jellies, paints, powders, and aerosols.

Encapsulating materials can also be employed with the compounds of the present invention, and the term “composition” can include the active ingredient in combination with an encapsulating material as a formulation, with or without other carriers. For example, the [1-(4-tert-Butylbenzyl)-5-(3-methylphenyl)1H-indol-3-yl](oxo)acetic acid polymorphs of the present invention can be delivered as microspheres for slow release in the body. In one embodiment, microspheres can be administered via intradermal injection of drug-containing microspheres, which slowly release the drug subcutaneously (see Rao, J. Biomater Sci. Polym. Ed. 7:623-645, 1995; as biodegradable and injectable gel formulations (see, e.g., Gao, Pharm. Res. 12:857-863, 1995); or, as microspheres for oral administration (see, e.g., Eyles, J. Pharm. Pharmacol. 49:669-674, 1997). Both transdermal and intradermal routes afford constant delivery for hours, days, weeks or months. Cachets can also be used in the delivery of the compounds of the present invention, e.g., anti-atherosclerotic medicaments.

In another embodiment, the [1-(4-tert-Butylbenzyl)-5-(3-methylphenyl)1H-indol-3-yl](oxo)acetic acid polymorphs of the present invention can be delivered by the use of liposomes which fuse with the cellular membrane or are endocytosed, i.e., by employing ligands attached to the liposome, or attached directly to the drug, that bind to surface membrane protein receptors of the cell resulting in endocytosis. By using liposomes, particularly where the liposome surface carries ligands specific for target cells, or are otherwise preferentially directed to a specific organ, one can focus the delivery of the compound into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989, the disclosures of which are herein incorporated by reference).

In other cases, the preferred preparation can be a lyophilized powder which may contain, for example, any or all of the following: about 1 mM-50 mM histidine, about 0.1%-2% sucrose, about 2%-7% mannitol, at a pH range of about 4.5 to about 5.5, that is combined with buffer prior to use.

A pharmaceutical composition of the invention can optionally contain, in addition to [1-(4-tert-Butylbenzyl)-5-(3-methylphenyl)-1H-indol-3-yl](oxo)acetic acid polymorphs, at least one other therapeutic agent useful in the treatment of a disease or condition associated with increased PAI-1 activity.

The pharmaceutical compositions are generally formulated as sterile, substantially isotonic and in full compliance with all Good Manufacturing Practice (GMP) regulations of the U.S. Food and Drug Administration.

### Dosage Regimens

The present invention provides methods of inhibiting PAI-1 activity in a subject for the treatment of diseases and conditions associated with increased PAI-1 activity using [1-(4-tert-Butylbenzyl)-5-(3-methylphenyl)1H-indol-3-yl](oxo)acetic acid polymorphs. In one embodiment of the present invention, a skilled practitioner can treat a subject having a disease associated with elevated PAI-1 levels and/or activity with [1-(4-tert-Butylbenzyl)-5-(3-methylphenyl)1H-indol-3-yl](oxo)acetic acid polymorphs of the present invention.

For treatment purposes, the [1-(4-tert-Butylbenzyl)-5-(3-methylphenyl)1H-indol-3-yl](oxo)acetic acid polymorphs disclosed herein can be administered to the subject in a single bolus delivery, via continuous delivery (e.g., continuous transdermal, mucosal, or intravenous delivery) over an extended time period, or in a repeated administration protocol (e.g., by an hourly, daily or weekly, repeated administration protocol). The pharmaceutical formulations of the present invention can be administered, for example, one or more times daily, 3 times per week, or weekly. In an exemplary embodiment of the present invention, the pharmaceutical formulations of the present invention are orally administered once or twice daily.

In this context, a therapeutically effective dosage of the biologically active agent(s) can include repeated doses within a prolonged treatment regimen that will yield clinically significant results to alleviate one or more symptoms or detectable conditions associated with increased PAI-1 activity. Determination of effective dosages in this context is typically based on animal model studies followed up by human clinical trials and is guided by determining effective dosages and administration protocols that significantly reduce the occurrence or severity of targeted exposure symptoms or conditions in the subject. Suitable models in this regard include, for example, murine, rat, porcine, feline, non-human primate, and other accepted animal model subjects known in the art. Alternatively, effective dosages can be determined using in vitro models (e.g., immunologic and histopathologic assays). Using such models, only ordinary calculations and adjustments are typically required to determine an appropriate concentration and dose to administer a therapeutically effective amount of the biologically active agent(s) (e.g., amounts that are intranasally effective, transdermally effective, intravenously effective, or intramuscularly effective to elicit a desired response). In alternative embodiments, an “effective amount” or “therapeutically effective dose” of the biologically active agent(s) will inhibit or enhance one or more selected biological activity(ies) correlated with a disease or condition, as set forth above, for either therapeutic or diagnostic purposes.

The actual dosage of biologically active agents will vary according to factors such as the extent of exposure and particular status of the subject (e.g., the subject's age, size, fitness, extent of symptoms, susceptibility factors, etc), time and route of administration, as well as other drugs or treatments being administered concurrently. Dosage regimens can be adjusted to provide an optimum prophylactic or therapeutic response. By “therapeutically effective dose” herein is meant a dose that produces effects for which it is administered. More specifically, a therapeutically effective dose of the compound(s) of the invention preferably alleviates symptoms, complications, or biochemical indicia of diseases associated with increased PAI-1 activity. The exact dose will depend on the purpose of the treatment, and will be ascertainable by one skilled in the art using known techniques (see, e.g., Lieberman, Pharmaceutical Dosage Forms (Vols. 1-3, 1992); Lloyd, 1999, The Art, Science, and Technology of Pharmaceutical Compounding; and Pickar, 1999, Dosage Calculations, the disclosure of which is herein incorporated by reference). A therapeutically effective dose is also one in which any toxic or detrimental side effects of the active agent is outweighed in clinical terms by therapeutically beneficial effects. It is to be further noted that for each particular subject, specific dosage regimens should be evaluated and adjusted over time according to the individual need and professional judgment of the person administering or supervising the administration of the compounds.

In an exemplary embodiment of the present invention, unit dosage forms of the compounds are prepared for standard administration regimens. In this way, the composition can be subdivided readily into smaller doses at the physician's direction. For example, unit dosages can be made up in packeted powders, vials or ampoules, and preferably in capsule or tablet form. The active compound present in these unit dosage forms of the composition can be present in an amount, for example, of from about one gram to about fifteen grams or more, for single or multiple daily administration, according to the particular need of the patient. By initiating the treatment regimen with a minimal daily dose of about one gram, the blood levels of PAI-1 and the patient's symptomatic relief analysis can be used to determine whether a larger or smaller dose is indicated. Effective administration of the compounds of this invention can be given at an oral dose of, for example, from about 0.1 mg/kg/day to about 1,000 mg/kg/day. Preferably, administration will be from about 10/mg/kg/day to about 600 mg/kg/day, more preferably from about 25 to about 200 mg/kg/day, and even more preferably from about 50 mg/kg/day to about 100 mg/kg /day.

### Kits

After a pharmaceutical composition comprising [1-(4-tert-Butylbenzyl)-5-(3-methylphenyl)-1H-indol-3-yl](oxo)acetic acid polymorphs has been formulated in a suitable carrier, it can be placed in an appropriate container and labeled for treatment of a PAI-1 related disorder, e.g., leukemia. Additionally, another pharmaceutical comprising at least one other therapeutic agent useful in the treatment of the PAI-1 related disorder can be placed in the container as well and labeled for treatment of the indicated disease. Alternatively, a single pharmaceutical comprising [1-(4-tert-Butylbenzyl)-5-(3-methylphenyl)1H-indol-3-yl](oxo)acetic acid polymorphs and at least one other therapeutic agent useful in the treatment of a PAI-1 related disorder can be placed in an appropriate container and labeled for treatment. For administration of pharmaceuticals comprising [1-(4-tert-Butylbenzyl)-5-(3-methylphenyl)1H-indol-3-yl](oxo)acetic acid polymorphs and of pharmaceuticals comprising, in a single pharmaceutical, [1-(4-tert-Butylbenzyl)-5-(3-methylphenyl)1H-indol-3-yl](oxo)acetic acid polymorphs and at least one other therapeutic agent useful in the treatment of a PAI-1 related disorder, such labeling would include, for example, instructions concerning the amount, frequency and method of administration. Similarly, for administration of multiple pharmaceuticals provided in the container, such labeling would include, for example, instructions concerning the amount, frequency and method of administration of each pharmaceutical.

Embodiments of this invention may be considered singly or in combination.

References disclosed herein are incorporated by reference in their entirety.

What is claimed:

1. A polymorph (Form A) of [1-(4-tert-Butylbenzyl)-5-(3-methylphenyl)1H-indol-3-yl](oxo)acetic acid having a powder X-ray diffraction pattern comprising characteristic peaks, in terms of 2θ, at about 6.5° and 10.9°.

2. The polymorph of claim 1 having a powder X-ray diffraction pattern further comprising characteristic peaks, in terms of 2θ, at about 18.6° and 24.2°.

3. The polymorph of claim 1 having a powder X-ray diffraction pattern further comprising characteristic peaks, in terms of 2θ, at about 16.2° and 17.4°.

4. The polymorph of claim 1 having a powder X-ray diffraction pattern further comprising characteristic peaks, in terms of 2θ, at about 15.2° and 25.8°.

5. The polymorph of claim 1 wherein said powder X-ray diffraction pattern comprises at least 5 additional characteristic peaks, in terms of 2θ, selected from 13.7°, 15.2°, 16.2°, 17.4°, 18.6°, 19.8°, 20.4°, 21.0°, 22.0°, 24.2°, and 25.8°.

6. The polymorph of claim 1 having a powder X-ray diffraction pattern further comprising characteristic peaks, in terms of 2θ, at about 9.9°, 11.5°, 13.7°, 14.2°, 14.5°, 15.2°, 16.2°, 17.4°, 18.6°, 19.8°, 20.1°, 20.4°, 21.7°, 22.0°, 24.2°, 24.9°, 25.8°, 26.1°, and 27.5°.

7. The polymorph of claim 1 having an X-ray powder diffraction pattern substantially as shown in FIG. 1.

8. A polymorph (Form A) of [1-(4-tert-Butylbenzyl)-5-(3-methylphenyl)1H-indol-3-yl](oxo)acetic acid having a differential scanning calorimetry trace showing a melting peak at about 138° C.; having onset about 134° C.

9. A pharmaceutical composition comprising a polymorph of claim 1, wherein at least 3% by weight of total [1-(4-tert-Butylbenzyl)-5-(3-methylphenyl)-1H-indol-3-yl](oxo)acetic acid in said composition is said polymorph.

10. The composition of claim 9, wherein at least 50% by weight of total [1-(4-tert-Butylbenzyl)-5-(3-methylphenyl)1H-indol-3-yl](oxo)acetic acid in said composition is said polymorph.

11. The composition of claim 9, wherein at least 90% by weight of total [1-(4-tert-Butylbenzyl)-5-(3-methylphenyl)1H-indol-3-yl](oxo)acetic acid in said composition is said polymorph.

12. A method for treatment of:
(a) thrombosis or fibrinolytic impairment;
(b) myocardial ischemia;
(c) Alzheimer's disease;
(d) reducing amyloid beta levels;
(e) improving cognition;
(f) treating pre-senile or senile dementia; or
(g) treating amyotrophic lateral sclerosis;
in a mammal comprising the administration of an effective amount of compound according to claim 1 to a mammal in need thereof.

13. The method of claim 12, wherein the said thrombosis or fibrinolytic impairment is associated with formation of atherosclerotic plaques, venous or arterial thrombosis, myocardial ischemia, atrial fibrillation, deep vein thrombosis, coagulation syndromes, pulmonary fibrosis, cerebral thrombosis, thromboembolic complications of surgery or peripheral arterial occlusion.

14. The method of claim 12, wherein amyloid beta levels are reduced in the brain.

15. The method according to claim 12, wherein said mammal is a human.

16. A method of preparing a polymorph of claim 1 comprising re-crystallization of [1-(4-tert-Butylbenzyl)-5-(3-methylphenyl)1H-indol-3-yl](oxo)acetic acid in one or more aromatic hydrocarbons, one or more polar non-protic solvents, or one or more dialkylethers, or combinations thereof.

17. The method of claim 16 wherein said aromatic hydrocarbon comprises toluene; and said dialkylethers comprises of t-butyl methyl ether; and said polar non-protic solvents comprises acetonitrile.

18. A method of preparing a polymorph of claim 1 comprising: treatment of [1-(4-tert-Butylbenzyl)-5-(3-methylphenyl)1H-indol-3-yl](oxo)acetic acid a) by heating in acetonitrile, cooling to from about 15° C. to about 30° C., treating with water and collecting the polymorph of claim 1; or b) by heating in toluene, addition of a non-aromatic hydrocarbon and collecting the polymorph of claim 1; or c) by heating in t-butyl methyl ether at a temperature sufficient for dissolution, addition of a non-aromatic hydrocarbon, seeding with polymorph of claim 1, and collecting the polymorph of claim 1.

19. The method of claim 18 c) wherein additional non-aromatic hydrocarbon is added after seeding followed by cooling of the solution to a temperature between about 15° C. to about 30° C.

20. A polymorph (Form B) of [1 -(4-tert-Butylbenzyl)-5-(3-methylphenyl)1H-indol-3-yl](oxo)acetic acid having a powder X-ray diffraction pattern comprising characteristic peaks, in terms of 2θ, at about 5.2° and 25.6°.

21. The polymorph of claim 20 having a powder X-ray diffraction pattern further comprising characteristic peaks, in terms of 2θ, at about 15.5° and 16.1°.

22. The polymorph of claim 20 having a powder X-ray diffraction pattern further comprising characteristic peaks, in terms of 2θ, at about 10.8 and 15.2°.

23. The polymorph of claim 20 having a powder X-ray diffraction pattern further comprising characteristic peaks, in terms of 2θ, at about 23.0°, 23.6°, 24.3° and 26.6°.

24. The polymorph of claim 20 wherein said powder X-ray diffraction pattern comprises at least 5 additional characteristic peaks, in terms of 2θ, selected from 10.8°, 15.2°, 15.5°, 16.1°, 23.0°, 23.6°, 24.3° and 26.6°.

25. The polymorph of claim 20 having a powder X-ray diffraction pattern further comprising characteristic peaks, in terms of 2θ, at about 10.3°, 10.8°, 11.1°, 15.2°, 15.5°, 16.1°, 16.4°, 16.6°, 17.0°, 17.4°, 18.7°, 19.5°, 19.7°, 21.0°, 21.6°, 22.3°, 23.0°, 23.6°, 24.3°, 24.6°, 26.6°, 28.4°, 28.7°, 29.5°, 30.4°, 30.7°, 31.4°, 31.7° and 35.9°.

26. The polymorph of claim 20 having an X-ray powder diffraction pattern substantially as shown in FIG. 6.

27. A polymorph (Form B) of [1 -(4-tert-Butylbenzyl)-5-(3-methylphenyl)1H-indol-3-yl](oxo)acetic acid having a differential scanning calorimetry trace showing a melting peak at about 131° C.; having onset about 128° C.

28. A pharmaceutical composition comprising a polymorph (Form B) of claim 20, wherein at least 3% by weight of total [1-(4-tert-Butylbenzyl)-5-(3-methylphenyl)-1H-indol-3-yl](oxo)acetic acid in said composition is said polymorph.

29. The composition of claim 28 wherein at least 50% by weight of total [1-(4-tert-Butylbenzyl)-5-(3-methylphenyl)1H-indol-3-yl](oxo)acetic acid in said composition is said polymorph.

30. The composition of claim 28 wherein at least 90% by weight of total [1-(4-tert-Butylbenzyl)-5-(3-methylphenyl)1H-indol-3-yl](oxo)acetic acid in said composition is said polymorph.

31. A method for treatment of:
(a) thrombosis or fibrinolytic impairment;
(b) myocardial ischemia;
(c) Alzheimer's disease;
(d) reducing amyloid beta levels;
(e) improving cognition;
(f) treating pre-senile or senile dementia; or
(g) treating amyotrophic lateral sclerosis;
in a mammal comprising the administration of an effective amount of compound according to claim 20 to a mammal in need thereof.

32. The method of claim 31, wherein the said thrombosis or fibrinolytic impairment is associated with formation of atherosclerotic plaques, venous or arterial thrombosis, myocardial ischemia, atrial fibrillation, deep vein thrombosis, coagulation syndromes, pulmonary fibrosis, cerebral thrombosis, thromboembolic complications of surgery or peripheral arterial occlusion.

33. The method of claim 31, wherein amyloid beta levels are reduced in the brain.

34. The method according to claim 31 wherein said mammal is a human.

35. A method of preparing a polymorph of claim 20 comprising dissolution of [1-(4-tert-Butylbenzyl)-5-(3-methylphenyl)1H-indol-3-yl](oxo)acetic acid in one or more aromatic hydrocarbons followed by evaporation of solvent.

36. The method of claim 35 wherein the aromatic hydrocarbon comprises toluene.

37. A polymorph (Form C) of [1-(4-tert-Butylbenzyl)-5-(3-methylphenyl)1H-indol-3-yl](oxo)acetic acid having a powder X-ray diffraction pattern comprising characteristic peaks, in terms of 2θ, at about 5.4° and 6.8°.

38. The polymorph of claim 37 having a powder X-ray diffraction pattern further comprising characteristic peaks, in terms of 2θ, at about 9.9° and 13.5°.

39. The polymorph of claim 37 having a powder X-ray diffraction pattern further comprising characteristic peaks, in terms of 2θ, at about 6.0° and 16.2°.

40. The polymorph of claim 37 having a powder X-ray diffraction pattern further comprising characteristic peaks, in terms of 2θ, at about 13.2°, 17.3°, 19.9° and 20.6°.

41. The polymorph of claim 37 wherein said powder X-ray diffraction pattern comprises at least 5 additional characteristic peaks, in terms of 2θ, selected from 6.0°, 9.9°, 13.2°, 13.5°, 16.2°, 17.3°, 19.9° and 20.6°.

42. The polymorph of claim 37 having a powder X-ray diffraction pattern further comprising characteristic peaks, in terms of 2θ, at about 6.0°, 9.9°, 10.9°, 13.2°, 13.5°, 16.2°, 17.0°, 17.3°, 19.5°, 19.9°, 20.6°, 21.2°, 22.120 , 23.0° and 24.5°.

43. The polymorph of claim 37 having an X-ray powder diffraction pattern substantially as shown in FIG. 8.

44. A polymorph (Form C) of [1-(4-tert-Butylbenzyl)-5-(3-methylphenyl)1H-indol-3-yl](oxo)acetic acid having a differential scanning calorimetry trace showing a melting peak at about 82° C.; having onset about 74° C.

45. A pharmaceutical composition comprising a polymorph (Form C) of claim 37, wherein at least 3% by weight of total [1-(4-tert-Butylbenzyl)-5-(3-methylphenyl)-1H-indol-3-yl](oxo)acetic acid in said composition is said polymorph.

46. The composition of claim 45 wherein at least 50% by weight of total [1-(4-tert-Butylbenzyl)-5-(3-methylphenyl)1H-indol-3-yl](oxo)acetic acid in said composition is said polymorph.

47. The composition of claim 45 wherein at least 90% by weight of total [1-(4-tert-Butylbenzyl)-5-(3-methylphenyl)1H-indol-3-yl](oxo)acetic acid in said composition is said polymorph.

48. A method for treatment of:
(a) thrombosis or fibrinolytic impairment;
(b) myocardial ischemia;
(c) Alzheimer's disease;
(d) reducing amyloid beta levels;
(e) improving cognition;
(f) treating pre-senile or senile dementia; or
(g) treating amyotrophic lateral sclerosis;
in a mammal comprising the administration of an effective amount of compound according to claim 37 to a mammal in need thereof.

49. The method of claim 48, wherein the said thrombosis or fibrinolytic impairment is associated with formation of atherosclerotic plaques, venous or arterial thrombosis, myocardial ischemia, atrial fibrillation, deep vein thrombosis, coagulation syndromes, pulmonary fibrosis, cerebral thrombosis, thromboembolic complications of surgery or peripheral arterial occlusion.

50. The method of claim 48, wherein amyloid beta levels are reduced in the brain.

51. The method according to claim 48, wherein said mammal is a human.

52. A method of preparing a polymorph of claim 37 comprising dissolution of [1-(4-tert-Butylbenzyl)-5-(3-methylphenyl)1H-indol-3-yl](oxo)acetic acid in a solution comprising an alcohol followed by crystallization and collection of the polymorph.

53. The method of claim 52 wherein the solution comprising alcohol is heated to effect dissolution and cooled to effect crystallization.

54. The method according to claim 52 wherein the solution comprising alcohol is treated with a non-aromatic hydrocarbon counter solvent prior to collection of the polymorph.

55. The method according to claim 52 wherein the alcohol comprises ethanol; and the non-aromatic hydrocarbon counter solvent comprises heptane.
